(19) United States
(12) Patent Application Publication
(10) Pub. No.: US 2025/0215399 A1
(43) Pub. Date: Jul. 3, 2025

(54) SURFACE MODIFIED POLYMER SCAFFOLDS FOR REPAIRING CONNECTIVE TISSUE DEFECTS

(71) Applicant: The Board of Regents of the University of Oklahoma, Norman, OK (US)

(72) Inventors: Vassilios Sikavitsas, Norman, OK (US); Cortes Williams, III, San Antonio, TX (US)

(21) Appl. No.: 19/084,315

(22) Filed: Mar. 19, 2025

Related U.S. Application Data
(63) Continuation of application No. 16/630,799, filed on Jan. 13, 2020.
(60) Provisional application No. 62/532,823, filed on Jul. 14, 2017.

(51) Int. Cl. C12N 5/071; A61L 27/38; C12N 5/00

(52) U.S. Cl. CPC C12N 5/0697; C12N 5/0062; A61L 27/38; C12N 2533/40

(57) ABSTRACT

Polymer scaffolds for engineering tissue constructs for defect repair and testing are disclosed. The scaffolds comprise a high molecular weight polymer having an external surface functionalized with a low molecular weight polymer to which cell surface binding molecules are attached for enhancing cell adherence to the scaffold. In particular the tissue constructs may be used to repair connective tissue defects.

### CROSS-REFERENCE TO RELATED APPLICATIONS

The present application is continuation of U.S. Ser. No. 16/630,799, filed Jan. 13, 2020, now abandoned, which is a national stage application of International Application No. PCT/US2018/041972, filed Jul. 13, 2018, which claims priority to U.S. Provisional Application Ser. No. 62/532,823, filed Jul. 14, 2017, which claims the benefit under 35 U.S.C. 119(e). The disclosure of each of the above applications is hereby expressly incorporated herein by reference in its entirety.

### BACKGROUND

The available in vivo technology for testing of new drug therapies mostly relies on immunodeficient mouse models. These include using cancer cell lines or patient cells grown in a mouse, and administering the drug to the mouse once the tumor has matured. There are at least two disadvantages to this method. First the process is very expensive, and second, the tumor in the mouse does not behave as it would in a human. In vitro testing with techniques that correspond to in vivo results is obviously a goal. Although the funding for cancer research leads to many new drug therapy studies, the methods for testing these in vitro lags behind. Currently, a truly viable option for the in vitro testing of chemotherapeutic drug cocktails does not exist. Typically, during the initial evaluation process, new drugs are usually first tested in 2D in vitro cultures before moving on to 3D in vivo animal models. However, these drugs often do not perform the same in 2d as they do in 3D. This is mainly due to how differently cancer cells behave in 3D, where they express different surface receptors, proliferation rates, and metabolic functions. Therefore, a major problem that faces the industry is the creation of in vitro models that can closely align with in vivo conditions, and also the ability to grow large tumors. Due to this, 3D in vitro models are an important step between 2D in vitro cultures and 3D in vivo cultures. By first testing drugs in a 3D in vitro model, researchers would gain better insight as to how the drug will affect 3D tumors before moving on to testing in more costly and lengthy-term in vivo animal models. Such a 3D culture system could also be used for patient-specific growth of tumors in an in vitro environment, enabling the testing of patient-specific treatment modes that could be tested on the patient's own cells before using the treatment mode in the patient him/herself, thus avoiding unnecessary harm and cost to the patient. Such a system would allow oncologists and researchers to save time and money in the search for compatible therapies, but also shield patients from multiple unsuccessful rounds of chemotherapy or treatments.

### BRIEF DESCRIPTION OF THE DRAWINGS

Several embodiments of the present disclosure are hereby illustrated in the appended drawings. It is to be noted however, that the appended drawings only illustrate several embodiments and are therefore not intended to be considered limiting of the scope of the present disclosure.

FIG. 1 shows a schematic of the linking of low molecular weight amine-terminated poly(L-lactic acid) (lowMW AT-PLLA) to a high molecular weight poly(L-lactic acid) (highMW PLLA) scaffold, followed by treatment with a linker molecule (e.g., N-succinimidyl 3-(2-pyridyldithio) propionate (SPDP)), followed by linkage to a binding ligand molecule (e.g., arginineglycineaspartic acid (RGD)).

FIG. 2 shows detection of pyridine-2-thione released during the modification process of differently treated PLLA scaffolds. When SPDP linked to the aminated surfaces is reacted with a secondary peptide it releases pyridine-2-thione, producing a wavelength having an absorbance at 405 nm. The results show that a lowMW AT-PLLA-aminated scaffold which is linker-modified with SPDP has the highest level of amine groups on the scaffold after treatment with RGD peptide.

FIG. 3 shows surface activity of aminated surfaces of PLLA over a one week period for surface modified versus bulk prepared methods. When NHS rhodamine is reacted with each construct prior to fluorescent imaging, it attaches to free amine groups, which can be quantified using fluorescent imaging. The intensity for the surface modified surfaces is about double that of bulk prepared surfaces after one week. Values are given as the mean±standard error of the mean (n=3).

FIG. 4 is a schematic of a flow perfusion apparatus system used in the present disclosure. The lefthand image shows an assembled perfusion apparatus, and the righthand image shows a disassembled apparatus.

FIG. 5 shows the effect of the extent of amine-terminated entrapment on mesenchymal stem cell surface area after linkage of arginineglycineaspartic acidcysteine (RGDC) peptides to amine groups entrapped in poly(L-lactic acid) discs. The concentrations listed are amount of RGD in solution that were available for reaction. Controls (bars with striped lines) indicate scaffolds that were amine-modified only. Plain represents baseline absorbance of PLLA. Values are given as the mean±standard error of the mean (n=3). Significance is calculated via ANOVA with Tukey HSD Post-hoc analysis.

FIG. 6 shows that cell spreading, or the actin surface area covered per cell, is an indicator of the extent of cellular adhesion strength. Cell spreading, or the actin surface area covered per cell, is shown for B16 cancer cells seeded on (1) plain non-modified highMW PLLA films, (2) highMW PLLA films modified to express n-cadherin using amine-amine coupling, and (3) highMW PLLA films modified to using amine-carboxyl binding. Values are given as the mean±standard error of the mean (n=3).

FIG. 7 shows seeding efficiency of various cell types on PLLA scaffolds. Designation “(mod)” indicates cells were seeded on scaffolds that have been modified with (coated) RGD peptide using amine-amine coupling (MSCs) or N-cadherin using amine-carboxyl binding (B16). Values are given as the mean±standard error of the mean (n=3). Significance is indicated by * (p<0.01), and was calculated via ANOVA with Tukey HSD Post-hoc analysis.

### DETAILED DESCRIPTION

Compositions and methods are disclosed for producing polymer scaffolds for tissue engineering and drug testing. The scaffolds comprise a high molecular weight polymer having an external surface functionalized with a low molecular weight polymer to which cell surface binding molecules are attached for enhancing cell adherence to the scaffold. In at least certain embodiments, the present disclosure is directed to methods of forming scaffolds for use in tissue engineering and for making implants that can be placed into an implant site for repairing a connective tissue defect thereby enabling healing and regeneration at the implant site. In certain non-limiting embodiments, the scaffold is used for bone, cartilage, tendon, and/or meniscus regeneration and repair. In certain non-limiting embodiments, the presently disclosed scaffolds can be used to repair osteochondral defects found in joint disorders, such as defects in articular cartilage and/or the subchondral bone in joints and joint structures. An implant site of the compositions of the present disclosure may be located in, but is not limited to, a bone, knee, ankle, elbow, shoulder, wrist, hip, vertebra, vertebral disc, patella, femoral head, glenoid of the scapula, growth plate, tendon, ligament, trachea, vocal cord, bronchus, fascia, and craniofacial bones such as the calvarium. In alternate embodiments, the scaffolds can be used for culturing cells and tumors which mimic the three-dimensional conditions in vivo, thereby providing improved method for drug testing and screening.

Before further describing various embodiments of the compositions and methods of the present disclosure in more detail by way of exemplary description, examples, and results, it is to be understood that the embodiments of the present disclosure are not limited in application to the details of methods and compositions as set forth in the following description. The embodiments of the compositions and methods of the present disclosure are capable of being practiced or carried out in various ways not explicitly described herein. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description. While the compositions and methods of the present disclosure have been described in terms of particular embodiments, it will be apparent to those of skill in the art that variations may be applied to the compositions and/or methods and in the steps or in the sequence of steps of the method described herein without departing from the concept, spirit, and scope of the inventive concepts as described herein. All such similar substitutes and modifications apparent to those having ordinary skill in the art are deemed to be within the spirit and scope of the inventive concepts as disclosed herein.

All patents, published patent applications, and non-patent publications referenced or mentioned in any portion of the present specification, including U.S. Ser. No. 62/532,823, filed on Jul. 14, 2017, are indicative of the level of skill of those skilled in the art to which the present disclosure pertains, and are hereby expressly incorporated by reference in their entirety to the same extent as if the contents of each individual patent or publication was specifically and individually incorporated herein.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the term “about” is used to indicate that a value includes the inherent variation of error for the composition, the method used to administer the composition, or the variation that exists among the objects, or study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately”, where used herein when referring to a measurable value such as an amount, percentage, temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. The range 100 units to 2000 units therefore refers to and includes all values or ranges of values of the units, and fractions of the values of the units and integers within said range, including for example, but not limited to 100 units to 1000 units, 100 units to 500 units, 200 units to 1000 units, 300 units to 1500 units, 400 units to 2000 units, 500 units to 2000 units, 500 units to 1000 units, 250 units to 1750 units, 250 units to 1200 units, 750 units to 2000 units, 150 units to 1500 units, 100 units to 1250 units, and 800 units to 1200 units. Any two values within the range of about 100 units to about 2000 units therefore can be used to set the lower and upper boundaries of a range in accordance with the embodiments of the present disclosure.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability of an active agent to modify the physiological system of an organism without reference to how the active agent has its physiological effects.

As used herein, “pure,” “substantially pure,” or “isolated” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species (e.g., the peptide compound) is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure. Where used herein the term “high specificity” refers to a specificity of at least 90%, or at least 91%, or at least 92%, or at least 93%, or at least 94%, or at least 95%, or at least 96%, or at least 97%, or at least 98%, or at least 99%. Where used herein the term “high sensitivity” refers to a sensitivity of at least 90%, or at least 91%, or at least 92%, or at least 93%, or at least 94%, or at least 95%, or at least 96%, or at least 97%, or at least 98%, or at least 99%.

The terms “subject” and “patient” are used interchangeably herein and will be understood to refer an organism to which the compositions of the present disclosure are applied and used, such as a vertebrate or more particularly to a warm blooded animal, such as a mammal or bird. Non-limiting examples of animals within the scope and meaning of this term include dogs, cats, rats, mice, guinea pigs, chinchillas, rabbits, horses, goats, cattle, sheep, llamas, zoo animals, Old and New World monkeys, non-human primates, and humans.

“Treatment” refers to therapeutic treatments, such as for bone defect healing. The term “treating” refers to administering the composition to a patient such therapeutic purposes, and may result in an amelioration of the condition or disease.

The terms “therapeutic composition” and “pharmaceutical composition” refer to an active agent composition, such as the hydrogel precursor and hydrogel compositions described herein, that may be administered to a subject by any method known in the art or otherwise contemplated herein, wherein administration of the composition brings about a therapeutic effect as described elsewhere herein. In addition, certain compositions of the present disclosure may be designed to provide delayed, controlled, extended, and/or sustained release using formulation techniques which are well known in the art.

The term “effective amount” refers to an amount of an active agent which is sufficient to exhibit a detectable biochemical and/or therapeutic effect, for example without excessive adverse side effects (such as toxicity, irritation and allergic response) commensurate with a reasonable benefit/risk ratio when used in the manner of the present disclosure. The effective amount for a patient will depend upon the type of patient, the patient's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by a person of ordinary skill in the art using routine experimentation based on the information provided herein.

The term “ameliorate” means a detectable or measurable improvement in a subject's condition or or symptom thereof. A detectable or measurable improvement includes a subjective or objective decrease, reduction, inhibition, suppression, limit or control in the occurrence, frequency, severity, progression, or duration of the condition, or an improvement in a symptom or an underlying cause or a consequence of the condition, or a reversal of the condition. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a condition, or consequences of the condition in a subject.

A decrease or reduction in worsening, such as stabilizing the condition, is also a successful treatment outcome. A therapeutic benefit therefore need not be complete ablation or reversal of the condition, or any one, most or all adverse symptoms, complications, consequences or underlying causes associated with the condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition (e.g., stabilizing), over a short or long duration of time (e.g., seconds, minutes, hours).

Returning to discussion of particular embodiments of the present disclosure, within biomedical engineering, the field of tissue engineering primarily addresses shortcomings in tissue damage repair and organ transplants. By developing techniques for in vitro tissue culture and development, tissue engineering presents solutions to tissue damage-related problems which are unavailable to purely in vivo treatments. In many cases, treatment for tissue damage involves direct autologous transplant of a similar tissue from another region within the body. This approach repairs critically damaged tissue at the cost of tissue strength and integrity from the donor site. The tissue engineering approach instead extracts adult stem cells from the host nondestructively. These cells are then cultured in vitro with scaffolding, media, and flow stresses which mimic an in vivo environment, allowing the stem cells to proliferate and then differentiate into cell types which effect the desired tissue. Once properly developed, the tissue matrix is reinserted at the location requiring tissue repair, providing both material to provide support and cells optimized to mend the damaged tissue.

Bone tissue engineering utilizes mesenchymal stem cells (MSC) derived from bone marrow extract to produce osteoblasts, a key component in bone development and maintenance. Osteoblastic tissue constructs developed in vitro will be used as grafts for delayed union or nonunion bone fractures. Currently, about 1 million delayed union or nonunion fractures occur in the U.S. each year. Standard treatment for these conditions involve an autologous bone graft, usually bone material removed from the hip. This method creates significant risk of morbidity and infection at the donor site as well as reducing the mechanical integrity of the hip bone. Development of a similar graft from MSCs would circumvent these complications.

The properties of the scaffold which supports the stem cells play a significant role in cell growth and differentiation and tissue development. As the supporting structure, the scaffold needs an appropriate degree of rigidity for the target tissue. Bone tissue in particular requires rigid scaffolding. Additionally, scaffolding requires porosity or maximal surface area for nutrient transfer and cell expansion. Scaffolds should also promote cell adhesion or they would not be viable surfaces for cell culture. When exposed to the tissue's native conditions, the scaffold should degrade at a rate similar to tissue development into a non-toxic product. These and other necessary and optimizable constraints place scaffold design as a central aspect of tissue engineering.

It is well-documented that inoculation of MSCs onto poly (L-lactic acid) (PLLA) scaffolds has resulted in cellularity and cell development. Static seeding efficiencies average only about 10% cell adherence. Methods of flow perfusion of cells suspended in media through 3-D scaffolds increase efficiencies to 30-40%. Though a significant improvement over static seeding, this approach requires extensive seeding periods and complex bioreactor design. The majority of cells are still flushed away without adhering. Secondarily, due to an adjustment period known as the “lag phase,” cell number drops over the first 24 hours as pioneer cells dedicate more energy to developing extracellular matrix (ECM). While the establishment of ECM allows surviving cells to grow more easily with recognizable morphologies, to energy cost of producing the ECM upon adhering to a novel surface extends the timeline between marrow extraction and fracture treatment.

Modification of the scaffold to create a surface which enhances cellular adherence and extracellular matrix generation directly addresses these limitations. These surfaces contain ECM molecules and derivatives which have been found to improve cell recognition of the surface and present a hydrophilic interface for cells. Although several methods for surface modification have been tested, plasma treatment has been the primary method. However, this method has difficulty in modifying internal surfaces in a 3D scaffold, and plasma exposure may alter mechanical properties of the underlying PLLA. These limitations render the plasma treatment method incompatible with 3D-scaffold flow perfusion bioreactors which subject the scaffold and cells to shear stresses similar to those present in bone structures. If a method for surface modification could attach biomolecules via surface interactions with a liquid solution rather than plasma exposure, then the surfaces could be biomolecule-functionalized without mechanical alteration of the interior PLLA structure. It is to such scaffold compositions and methods of their use in therapies and drug development that the present disclosure is directed.

Tissue engineering is a developing field that links biologics with engineering to promote tissue regeneration. Key components for successful tissue engineering is to have an appropriate cell or stem cell source typically derived from the patient via a cell biopsy. A scaffold that is biocompatible and bioabsorbable is seeded with the cells then applied a mechanical or chemical stimuli and growth factors that differentiate the cells on the construct into the accurate cell lineage. When implanted in a patient, the tissue construct is expected to influence extracellular matrix organization and construct degradation, limit any immune reactions, all while the native tissue remodels and regenerates. Tissue engineering has become a very popular method when combined with bioreactors for treating disorders of the musculoskeletal system.

Currently there are four main types of bone grafts used in therapeutic applications. The first three are natural: autografts, allografts, and xenografts, and the fourth is a synthetic graft material, such as bone cement. Autografts comprise healthy tissue taken from the patient's own body, and overall are the best type of bone graft, due to the fact immune rejection is not a serious factor. In this type of procedure, healthy tissue is taken from another site on the patient's body and is then transplanted to the desired area. However, these grafts suffer from a limited supply and also site morbidity and pain in the harvesting site. In addition, these grafts may fail due to many cells not surviving the transplantation process. Allografts comprise taken from a donor of the same species, and are another frequently used bone replacement. It has become more common in the past decade through the introduction of immunosuppressant drugs that help ease the immune response from foreign tissue entering the body. However, these grafts still cause immune rejection and have limited osteoinductive abilities when compared to autologous grafts. Another graft source are xenografts, which are tissues taken from a different animal species than the one receiving the graft. The most common animals used for such transplants are pigs, sheep, and goats. Unfortunately they carry a high rate of infection and host rejection. Due to this, xenogeneic grafts are not highly desired.

The fourth type of graft material is synthetic. Because natural tissue sources are difficult to come by, many people choose to get mechanical replacements. Hydroxyapatite is a synthetic bone substitute that has been frequently used. It is a brittle material that slowly undergoes bone resorption. Due to this, it is more often than not combined with other materials to increase the speed of resorption. Another common material is ceramics. They are usually made from tricalcium phosphate and have been shown to have osteogenic capabilities when they are attached to healthy bone. Compared to hydroxyapatite, ceramics have faster bone resorption, but must also be removed as the new bone grows.

When the whole bone has not been damaged, bone cements are often used. Polymethyl methacrylate (PMMA) is a popular bone cement that works by acting as an anchor, connecting bone to bone or bone to joint, and absorbing force in the same fashion natural bone would. The use of bone cement is very popular due to its widespread use and effectiveness. A disadvantage of bone cement is that it is non-biodegradable and thus permanent. It is also exothermic during polymerization and the heat produced during the reation is harmful to the neighboring tissue at the implantation site.

### Bone Tissue Engineering

Every year in the United States, there are more than 500,000 bone graft surgeries, with the most common needed for regenerating bone in fractural healing. In most cases, bone will regenerate after a fracture with minimal complications; however, when there is a critical-sized defect or fracture healing is impaired, bone grafts must be used to regain proper bone function. Furthermore, bone diseases such as osteoporosis, infection, skeletal defects, and bone cancer may also cause a need for bone grafts. Bone tissue engineering is a possible solution to the problems plaguing the current bone graft therapies. Because tissue engineered bone could be made of the patient's own cells, immune rejection would be eliminated as well as low availability. For this to work, four components are needed for tissue growth: cells that can be differentiated into bone cells, osteoconductive scaffolds for acting as a matrix while the tissue grows, growth factors and other chemical stimulation, and mechanical stimulation to encourage osteogenic differentiation. Mechanical stimulation is implemented through the use of bioreactors.

Bone is made of tightly packed collagen fibrils, which together form the lamellae. These collagen fibrils are what give compact bone its strength. By weight, compact bone contains approximately 30% matrix and 70% salt deposit. The organic matrix consists of over 90% collagen fibrils and the rest is ground substance, which is formed from the non-fibrous portions of extracellular matrix. Ground substance, for the most part, does not contain collagen, but it is made up of glycosaminoglycans, glycoproteins, and proteoglycans. Due to the orientation of the collagen fibers, along the direction of the force acting on the bones, the bone has a very high tensile strength compared to the other tissues in the body. The bone salts contained in compact bone are primarily calcium and phosphates. During calcification these two molecules form a crystalline salt known as hydroxyapatite. Spongy bone, or cancellous bone, is more loosely packed and has an average porosity of 30-90%. Cortical and spongy bone also differ in their function. Compact bone is more rigid, providing the structural strength, while the spongy bone provides the components for metabolic maintenance. The orientation of long bone is such that it can withstand the greatest amount of force. The inner section, diaphysis, contains a higher amount of compact bone meaning that it is made of tightly packed collagen fibrils. The epiphysis, ends of the bone, is wider than the diaphysis and contains a higher amount of spongy bone.

When cells are being cultured for use in bone tissue engineering, the marker for differentiation into bone cells is calcification. The first step includes both collagen monomers and ground substance being secreted by osteoblasts. These collagen monomers form collagen fibers that, in this early state, are called osteoids. Osteoids are similar to cartilage however the rate at which calcium precipitates in it is significantly higher. During the formation of osteoids, many osteoblasts become entrapped, and are from then on known as osteoclasts. Over the next few months, calcium salts form on the collagen fibers of the osteoid, and in time they undergo substitution becoming complete hydroxyapatite crystals. The calcium salts that are not converted into hydroxyapatite stay on the fibers as amorphous salts that can easily be released into the extracellular fluid.

Another important feature of bone is how it is continually being renewed. There are two process involved in this: deposition and absorption. Deposition is the act of osteoblasts continually calcifying bone, and absorption is the process of osteoclasts removing bone.

Native bone contains three cell types relevant to tissue engineering: osteoblasts, osteoclasts, and osteocytes. Another important bone cell type involved in bone tissue engineering is the osteoprogenitor cell. For tissue engineering applications, osteoprogenitor cells mainly differentiate from mesenchymal stem cells. These osteoprogenitor cells are the precursors for osteoblasts, osteocytes, and the bone lining cells; whereas the osteoclasts are formed through the fusion of mononuclear precursors, such as those from hemopoietic tissue.

Osteoblasts are the major cell type responsible for bone deposition, the growing of bone. Their main function is the development of mineralized tissue, which contains several proteins, such as osteocalcin and osteopontin, and collagenases that aid in osteoclast activation. Osteocalcin is a noncollagenous protein, created solely by osteoblasts, that is involved in controlling the rate of bone formation and bone mineral maturation. Since it is only secreted by osteoblasts, osteocalcin is a prime candidate for identifying if a stem cell culture is turning osteogenic, and is commonly used as a biochemical marker for bone formation. Osteopontin is a noncollagenous glycoprotein that is responsible for osteoclast attachment and resorption.

Osteocytes are formed from osteoblasts that have been entrapped in the bone matrix, and take care of the maintenance of the bone. Each osteocyte resides in its own space in the bone matrix, named lacunae and canaliculi, and are interconnected through channels. These channels, also known as gap junctions, serve as the passageway through which nutrients can be exchanged with between osteocytes, blood vessels, and other places throughout the bone.

Osteoclasts mainly function to resorb bone. On their membrane, they have both a smooth surface that serves as a connective area for attaching to the bone matrix using integrins and, as previously mentioned, the aid of matrix proteins such as osteopontin. They also have a rough surface where bone resorption takes place. Proteolytic enzymes and acids released from this rough border break down the bone by breaking down the organic matrix and bone mineral.

The osteoblastic cell source is a very vital part of the equation. The ideal cells for use in vitro have a high proliferation rate, an ability to differentiate into the cells necessary for the tissue to operate, and also the ability to deposit organic tissue matrix. The most popular cells that are considered for bone tissue engineering are mesenchymal stem cells (MSCs), adipose derived stem cells, osteoblastic progenitor cells, osteoblasts, and osteocytes. For the most part, osteoclasts have not been utilized since they are not required for the formation of mineralized tissue. MSCs are the most widely used cells for bone tissue engineering. MSCs have been found to have increased osteoblastic differentiation when exposed to fluid shear, and also have exhibited clear osteoinductive capabilities. Furthermore it is widely known that MSCs have a higher proliferation rate when compared to osteoblasts and osteocytes.

Scaffolds are a necessity for supporting cells in in vitro applications of tissue engineering. For bone tissue engineering, the scaffold must allow the cells to not only attach and proliferate, but must also allow for mechanical stimulation, encourage bone cell migration, act as a substrate for osteoid deposition, and deliver bioactive molecules. Another aspect that is desired is biodegradability. If a tissue engineered construct is put into the body, the scaffold must degrade over time leaving only organic material that will retain natural levels of mechanical strength. The common scaffold types used for bone growth are natural polymers (fibrin and collagen), synthetic polymers (polycarbonates, polyanhidrides, poly(ethylene oxide), polyfumarates, and polyphosphazene), metals and ceramics. Examples of biodegradable polymers which have been used include poly(lactic acid) (PLA), poly(glycolic acid) (PGA), and poly(lactic-co-glycolic acid) (PLGA).

Currently, there are two major types of scaffolds: injectable and prefabricated. Injectable scaffolds are desirable due to their ability to assume the shape of highly irregular bone defect sites. However, a major big problem currently facing injectable scaffolds is their inability to generate controllable porous networks that can be infiltrated by host osteoprogenitors and vascular network. Without a vascular network, cells that do reside in the center of the construct will not be able to obtain optimal oxygen and other nutrient supplies. In addition, very carefully designed crosslinking strategies are required to avoid the release undesirable chemicals or heat that may harm the host neighboring host tissue. Prefabricated scaffolds, on the other hand, have highly controllable porosity that will allow enhanced levels of nutrient delivery throughout the construct, and also the possibility for higher mechanical strength. Common types of scaffolds include fiber meshes (woven and nonwoven), porous foams (salt leached or gas foamed), and those made from rapid prototyping such as 3D printing.

Cell culturing for bone tissue engineering is very similar to that for other tissue engineering fields. It is necessary to have a culture media, such as, for example, alpha-minimal essential media, d-minimal essential media, or F12, which contains the proper nutrients for cell growth. These media often include d-glucose, l-glutamine, HEPES (a buffer for maintaining physiological pH), phenol red indicator (for easily identifying pH). The differences between culture media are usually in their concentration of glucose, growth factors, and other nutrients. Another commonly used component of cell culture media is fetal bovine serum (FBS), newborn calf serum (NBCS), or some other similar animal blood serum. The purpose of these is to provide the cells with the proper growth factors to facilitate cell growth. It is important to note, however, that sera must always be screened to insure that they will differentiate cells properly, due to the fact serum concentrations are not consistent between batches.

When bone cells are desired, osteogenic media is used. Osteogenic media differs from regular culture media in that it contains dexamethasone, beta-glycerophosphate, and ascorbic acid. Dexamethasone is a glucocorticoid that has been found to facilitate bone differentiation and mineralization in cultures. Beta-glycerophosphate and ascorbic acid roles are to provide phosphate and increase collagen fibril production, respectively. Growth factors may also be added to aid in osteogenic differentiation. For example, bone morphogenetic proteins (BMPs), such as BMP-2 and BMP-7, are osteoinductive growth factors that are commonly used as growth factors to enhance mesenchymal stem cell differentiation into osteoblasts. BMP-2 is an important growth factor as it is involved in the TGF beta-signaling pathway, and aids in osteogenesis, cell growth, and differentiation. Once the cells are ready to be used for experiment, they must be seeded onto the scaffolds. Although the procedure may vary, static and dynamic seeding are the two most commonly used seeding methods. Static seeding consists of injecting cells onto scaffolds that are sitting in a culture well plate. In dynamic seeding, the cells suspended in media are allowed to flow through the scaffold. In theory, this allows for a greater level of cell penetration.

Mechanical forces have a strong effect on the deposition and resorption of bone. As force is applied to the bone, a pressure change occurs which leads to fluid flow inside the lacunae and canaliculi. This of course means that bone cells in the body are constantly being subjected to flow. Studies have shown that cell growth is stimulated by flow induced shear in vitro. Shear stresses under 2.5 Pa have corresponded to an increase in cell proliferation and osteoblastic differentiation. On the hand, shear above 2.5 Pa can cause cells to detach from the scaffold matrix.

Before beginning to culture cells for bone tissue engineering, it is useful to pick the proper bioreactor. In vitro bone development benefits from fluid flow that provided mechanical stimulation, all while improving oxygen and nutrient delivery throughout the scaffold and removing cell waste. For most bioreactors in bone tissue engineering, the parameter that is important is the shear stress associated with this fluid flow. It has been widely seen that mechanical stimulation can affect ECM production, cell proliferation, ECM calcification, and osteogenic gene expression. In order to properly accomplish this, four different types of bioreactors are commonly used: static culture flasks, spinner flasks, rotating wall vessels, and flow perfusion reactors. The following are descriptions of common bioreactors used for bone tissue engineering.

In static culture flasks, cells are seeded onto a scaffold, usually by pipetting a cell suspension on the top surface, and the construct is placed in a flask. This flask is then filled with cell culture media and stored in an incubator, with media changes occurring intermittently. Static 3D cultures suffer from severe limitations due to the fact there is no fluid flow through the scaffold porosity, or even around it, and therefore concentration gradients of nutrients develop with potentially harmful results, while the absence of shear limits osteoblastic differentiation. Studies have shown that using 3D static culture for bone tissue engineering results in low seeding efficiency, nonhomogeneous cell distribution, unfavorable nutrient gradients, and slow ECM mineralization.

Spinner flask bioreactors comprise cell-seeded scaffolds suspended in media in a cylindrical vessel. The goal of this setup is to create convective flow around the scaffold, through the use a mechanical drive or a magnetic stir bar. The stirring motion creates localized shear around the scaffolds. The flow environment at the surface of the scaffolds can be turbulent and may contain eddies with potentially harmful results to cells residing at exterior surface of the scaffolds. These bioreactors have been shown to be an improvement over static cultures for seeding efficiency, cell proliferation, and differentiation, all of which are to be expected, since the presence of continuous mixing allows for the mitigation of nutrient concentration gradients. However, spinner flasks are unable to efficiently deliver any significant amount of nutrients throughout the 3D scaffold leading to cell death near the center of the scaffold.

Rotating wall bioreactors comprise a rotating outer cylinder, a stationary inner cylinder, and an area for the culture between them. The inner wall is gas permeable through which oxygen is supplied to the system, while the outer cylinder is impermeable to gases and induces the dynamic field. An advantage of the rotating wall vessels is their ability to generate highly controllable fluid flow environments without the generation of eddies near the scaffold surfaces. As such, this type of design is ideal for studies where tight control of surface stresses is required, but as with the spinner flask, the inability to provide any significant convection to the interior porosity limits their usefulness in bone tissue engineering studies.

Perfusion bioreactors have been shown to be the most effective systems for bone tissue engineering, and it has been found that they deliver a wide range of easily manipulated shear stresses to stimulate differentiation into bone cells. They usually comprise a pump, media reservoirs, scaffold chambers, and connecting tubes. There are two main types of perfusion bioreactors: scaffold perfusion, and perfusion column. Perfusion columns have space surrounding the scaffolds, through which the media can flow. By doing this, media is not required to flow throughout the scaffold and therefore the shear stresses are not as easily controlled, and nutrient delivery is lowered through the interior porosity of the scaffolds. Scaffold perfusion bioreactors give better control of shear stresses by flowing media directly through the scaffolds. This is accomplished by press-fitting the scaffold in a cassette that restricts fluid flow only through the pores of the scaffold. Assuming pore interconnectivity throughout the scaffold, fluid flow provides enhanced nutrient delivery to cells residing in all locations of the construct, and allow for better waste removal as well.

The main benefit for utilizing dynamic bioreactor culture is the mitigation of mass transport limitations. During extended culture, it is necessary to continually supply the cells with nutrients, such as oxygen and glucose, for them to continue to proliferate and differentiate. In static culture without media flow, nutrients are transported to the cells in the interior of the scaffold mainly by passive diffusion. Previous studies have shown that tissues above 600 μm suffer from large hypoxic areas. Therefore, only cells located near the periphery of the scaffold will have the nutrients required to proliferate. In order to culture constructs of clinically relevant size, it is necessary for bone tissue engineers to develop culture techniques that improve these undesirable nutrient gradients by adding convective mass transfer.

Perfusion bioreactors provide enhanced mass transport benefits. Due to the press fit cassettes, media is not only continuously supplied to the scaffold, but it also forced exclusively through the interior of the scaffold. By doing this, the nutrient gradient is mitigated, thus allowing cells to proliferate without experiencing severe hypoxic conditions. In addition to this, the unidirectional flow allows for continual waste removal from the scaffold

In the body, fluid flow induced shear forces give signals to mesenchymal stem cells to differentiate. As a force is applied to the bone, a pressure change occurs which leads to fluid flow inside the lacunae and canaliculi, leading to a signal transduction pathway resulting in these stem cells to differentiate into osteoblasts. In in vitro environments, previous research has shown shear stresses below 15 dynes/cm² to have a stimulatory effect promoting osteoblastic differentiation, matrix deposition and calcification, cell proliferation, and osteogenic gene expression in 3D constructs. Osteoblastic differentiation is more pronounced in scaffold perfusion systems. Due to media being forced through the interior of the scaffold, the average flow induced shear forces are more controllable and more evenly distributed throughout the scaffold.

Cell seeding is the first step in the development of in vitro bone tissue engineered constructs. Before a tissue can be grown, it is necessary to adhere cells to the surface of the scaffold, so that they may proliferate throughout the construct. In most cases, it is preferable to have a homogeneous seeding distribution so that the tissue can grow evenly across the entirety of the scaffold surface area. This is especially true in bone tissue engineering, where the cells must not only proliferate, but also deposit mineralized tissue. In terms of cellular adherence, this is measured in seeding efficiency, which is the number of cells adhered at the end of a seeding protocol in comparison to the number of cells that were initially placed on the construct.

In light of the aforementioned goals, static seeding is largely unsuitable. In static seeding, cells are suspended in media and either pipetted onto the surface of the scaffold or the scaffold is placed into the suspension. There are many techniques employed, such as vacuum evacuation or shaker plates, to induce cellular migration to the interior of the scaffold; however, the results suffer from inconsistencies and more often than usual demonstrate extended inhomogeneities that are clearly undesirable. Similar to the mass transport limitations that plague static cultures, cells to inhabit the periphery of the scaffold leading to nonhomogeneous tissue development.

In dynamic seeding, the cells are added to media, and fluid flow is utilized in the bioreactor system to help the cells navigate to the scaffold surface and potentially to the interior. The addition of flow produces higher seeding efficiency and a more homogenous cell distribution Although each of the discussed dynamic bioreactor designs may be used to accomplish this, the perfusion bioreactor is once again more efficient at completing this task. Oscillatory seeding comprises adding the cells to the media and then using a flow perfusion system to alternate the flow direction making sure that the residence time is adequate to permit cells to fully travel through the scaffold before flow direction is changed. By doing this, cells are forced back and forth, through the interior of the scaffold many times over. Using this method, seeding efficiencies are increased even more so than with unidirectional flow.

### Tumor Engineering

During the last decade, the number and effectiveness of in vitro cancer models have increased dramatically. Utilizing a variety of techniques for 3D culture, researchers have created models that more closely resemble and predict in vivo tumor drug responses; however, there is still more room for improvement. In particular, these in vitro models consistently exhibit poor cell proliferation and distribution, which severely limits their predictive capabilities. The number one hurdle that must be overcome is the development of tumor models that contain functional vascularization to not only delivery drugs, but also to deliver nutrients and reduce hypoxia. Work herein aims to alleviate these issues.

During the initial synthesis process, new drugs are usually first tested in 2D cultures before moving on to in vivo animal models; however, these drugs do not perform the same on 3D cultures as they do in 2D. This is mainly due to how differently cancer cells behave in 3D, where they express different surface receptors, proliferation rates, and metabolic functions. Due to this, 3D in vitro models are an important step between 2D and in vivo cultures. By first testing drugs in a 3D in vitro model, researchers can gain better insight as to how the drug will affect 3D tumors before moving on to more costly and lengthy animal models. Although the funding for cancer research leads to many new drug therapy studies, the methods for testing these in vitro lags behind. In particular, a major problem that faces the industry is the creation of in vitro models that can closely align with in vivo conditions, and also the ability to grow large tumors that contain functional vasculature.

Above all else, the phenomena of hypoxia is the major obstacle facing the growth of functional tissues larger than a few millimeters in vitro. In situ tumors characteristically exhibit hypoxic centers of mass, more dense around the outer edges. These hypoxic centers induce angiogenesis and the growth of the tissue. Engineered tumors, however, suffer from a lack of vascular growth during hypoxia. Due to this, the creation of proper vascularization in engineered tissues is of utmost importance to researchers. Significant progress has been made to alleviate this problem over the past five years with various methods being developed for testing. However the scalability of these methods is limited, negating their use in the creation of larger models. Additionally, they do not allow for intravenous drug testing; the main method of treating tumors in vivo. The development of constructs that can support this is necessary for more accurate in vitro drug response tests.

The ability to test drugs in vitro is a vital part of new cancer drug therapy development. It allows for a greater throughput than in vivo testing and is more cost effective. Though many systems have been proposed, there a few that are more widely utilized than others. Perhaps the most widely used in in vitro testing system is the use of multicellular cancer spheroids. These spheroids are large aggregates of cells and usually have diameters of around 250 μm. They have been widely used due to each individual spheroid representing a section of tissue that must be penetrated during drug delivery. The main problems with this method are that the spheroids are not capable of representing intravenous drug delivery, and each individual spheroid can only be made at a maximum of 600 μm as an increase leads to necrosis of the interior.

Another method gaining traction is the development of vascularized tumor models by using multicell sheet constructs. These constructs comprise many cell monolayers stacked on top of a resected vascular network. This method creates a perfusable system wherein the cell sheets are able to receive nutrients solely from the vessel network. The downside of this model is that the cell sheets are very thin (sometimes only a single cell thick), and stacking takes a considerable amount of time. Currently, only constructs that are a maximum of 12 sheets thick have been created, only a fraction of the in vivo equivalents.

Culturing tumor cells in 3D polymer scaffolds in bioreactors is directly analogous to tissue engineering studies, where stem cells are seeded on the scaffolds and cultured over a period of time allowing the cells to migrate, and proliferate throughout the construct. Perfusion bioreactors have proven to be a good method for growing these tumors due to the continuous introduction of nutrients into the system and subsequent removal of waste products. With this method comes the basic limitations of growing large tissues in vitro. Once the tumor gains in size, nutrients are no longer able to penetrate to the interior of the mass due to the newly formed tissue decreasing the construct porosity and a lack of vasculature to transport it into the interior. In the absence of oxygen, the interior of the mass dies from hypoxia, not unlike actual tumors. However in the case of tumors, this induces vessel formation and the eventual growth in size.

Additive manufacturing, commonly referred to as rapid prototyping or 3D printing, is a class of material fabrication characterized by the process it uses to create 3D models, i.e., building up layer by layer. The converse is subtractive manufacturing, which includes starting with a block material and removing, or milling, pieces bit by bit. Common forms of additive manufacturing include stereolithography, bioprinting, and fluid deposition modeling (FDM). In the interest of scaffold fabrication, the majority of these technologies have been utilized, but bioprinting and FDM have emerged as the top prospects. Bioprinting enables the creation of various scaffold architectures that are pre-seeded with cells, growth factors, or other additives which has been proven to be of great use for tissue culture. In terms of vascularization, however, current technologies only allow for either smaller scale microfluidic chambers to be created, or vessels that exhibit diameters and pore sizes much larger than in tumors.

FDM has been extensively as a fabrication tool in biomedical research mainly due to the ease of which it creates complicated 3D structures, including scaffolds, with PLLA. Various cell types have successfully been cultured on PLLA scaffolds created by FDM, some of them for extended periods of time. These scaffolds exhibit better mechanical integrity than those made with bioprinting, making it a better candidate for development of vascular tree networks that can deliver media throughout 3D constructs. It is important to note that these same properties make it difficult for cells to rearrange the fibers, which is an integral step in tissue growth and angiogenesis. Therefore a coupling of techniques, spunbonded fibers in addition to FDM fibers, can be used in this work. Current fabricated vessels tend to have vessel diameters of up to 500 μm and pore sizes as low as 10 μm. Though these have shown good results, the vessel dimensions do not replicate actual tumor vasculature. For prostate cancer for example, tumors have been shown to have vessel diameters of 20-30 μm, and pore cutoff sizes for general tumors are typically below 2 μm and average around 700 nm. However, recent advances in FDM such as better drivers and more accurate microstepping, will enable the creation of vessels having with greater resolution.

As previously stated, the vascularization of in vitro tissues is important for the success of the construct. To address this issue, vascularized cardiac tissue has been previously created by overlaying cellular sheets on top of a perfusable vascular bed. The vascular networks were resected from rat femoral muscles, decellularized, and then fixed in a custom-made one pass perfusion bioreactor. The bioreactor was created so that media was able to be fed into one side of the femoral vessel and out the other side. The cell sheets were made by co-culturing endothelial cells (EC) with cardiac cells in temperature responsive culture plates, which allowed for removal once full confluency was reached.

In at least certain embodiments, the films and scaffolds of the present disclosure may be composed of a low molecular weight (lowMW) polymer selected from the group consisting of polylactic acid (PLA), polyglycolic acid (PGA), poly caprolactone (PCL), poly (lactide-co-glycolide) (PLGA), poly (lactic acid/glycolic acid) (PLAGA), poly (lactide-co-caprolactone), poly (glycolide-co-caprolactone), poly (lactide-co-trimethylene carbonate), poly (glycolide-co-trimethylene carbonate), poly (lactide-co-glycolide-co-caprolactone), and poly (lactide-co-glycolide-co-trimethylene carbonate), and mixtures thereof.

In at least certain embodiments, the films and scaffolds of the present disclosure may be composed of a high molecular weight (highMW) polymer selected from the group consisting of polylactic acid (PLA), polyglycolic acid (PGA), poly caprolactone (PCL), poly (lactide-co-glycolide) (PLGA), poly (lactic acid/glycolic acid) (PLAGA), poly (lactide-co-caprolactone), poly (glycolide-co-caprolactone), poly (lactide-co-trimethylene carbonate), poly (glycolide-co-trimethylene carbonate), poly (lactide-co-glycolide-co-caprolactone), and poly (lactide-co-glycolide-co-trimethylene carbonate), and mixtures thereof.

The lowMW polymer and/or highMW polymer may be a PLA selected from the group consisting of poly (L-lactic acid) (PLLA), poly (D-lactic acid) (PDLA), poly (D/L-lactic acid) (PDLLA), and poly (L/D-lactic acid) (PLDLA), and mixtures thereof.

In at least certain embodiments, the films or scaffolds comprise, individually or in combination, a highMW version of at least one of PLA, PGA, PCL, and said copolymers thereof, and a lowMW version of at least one of PLA, PGA, PCL, and said copolymers thereof, which is at least partially water-soluble, and which is amine-terminated (AT) or carboxy-terminated (CT). In general, the high molecular weight polymer(s) of the present disclosure has an average MW of at least 5000 Da or greater (and is water-insoluble), and the low molecular weight polymer(s) has an average MW of less than 5000 Da (and is at least partially water soluble). As noted, the low MW polymers may be amine-terminated (AT-PLA) or carboxy-terminated (CT-PLA).

In at least certain embodiments, the average MW of the lowMW polymer used herein for bulk preparation or surface modification of the films or scaffolds is less than 1000 Da (e.g., 500 Da to 1000 Da), or less than 1250 Da, or less than 1500 Da, or less than 2000 Da, or less than 2250 Da, or less than 2500 Da, or less than 2750 Da, or less than 3000 MW Da, or less than 4000 MW Da, or less than 5000 Da, and is at least partially water-soluble.

In at least certain embodiments, the average MW of the highMW polymer molecules used herein for bulk preparation or for films or scaffolds that are surface modified is at least 5000 Da, at least 10,000 Da, at least 20,000 Da, at least 25,000 Da, at least 50,000 Da, at least 75,000 Da, at least 100,000 Da, at least 125,000 Da, at least 150,000 Da, at least 175,000 Da, at least 200,000 Da, at least 250,000 Da, at least 300,000 Da, at least 400,000 Da, at least 500,000 Da, at least 600,000 Da, at least 700,000 Da, at least 800,000 Da, at least 900,000 Da, or at least 1,000,000 Da.

Where the term “average” is used herein in reference to molecular weights of a polymer material, it is intended to refer to the average of molecular weights of a particular “type” of polymer sample. For example, a polymer designated as having a MW of 50,000 Da may comprise molecules having individual MW in a range of, for example, 20-80 kDa, or 40-60 kDa. Thus, the designated MW of a polymer sample actually refers to an average of a range of MWs in the sample.

In certain embodiments wherein a film or scaffold substantially comprising highMW polymer (e.g., highMW PLA) is surface modified (by solubilization) as described herein with a solution of lowMW amine- and/or carboxyl-terminated polymer (e.g., lowMW PLA), to form a treated film or scaffold (“treated material”), the treated material comprises (in wt %) a highMW polymer:lowMW polymer ratio of 99:1. In alternate embodiments, the treated material comprises (in wt %) a highMW polymer:lowMW polymer ratio of 99.9:0.1. In alternate embodiments, the treated material comprises (in wt %) a highMW polymer:lowMW polymer ratio of 99.99:0.01. In alternate embodiments, the treated material comprises (in wt %) a highMW polymer:lowMW polymer ratio of 99.999:0.001. In alternate embodiments, the treated material comprises (in wt %) a highMW polymer:lowMW polymer ratio of 99.9999:0.0001. In alternate embodiments, the treated material comprises (in wt %) a highMW polymer:lowMW polymer ratio of 99.99999:0.00001. In alternate embodiments, the treated material comprises (in wt %) a highMW polymer:lowMW polymer ratio of 99.999999:0.000001.

In at least certain embodiments wherein a film or scaffold substantially comprising highMW polymer is treated to be surface-modified (by solubilization) as described herein with a solution of lowMW amine- and/or carboxyl-terminated polymer (“lowMW polymer”), to form a treated film or scaffold (“treated material”), at least 75 wt %, to 80 wt %, to 85 wt %, to 90 wt %, to 95 wt %, to 98 wt %, to 99 wt % of the lowMW polymer in the treated material is spatially located within the upper 25%, by thickness, of the highMW polymer portion. In alternate embodiments, at least 75 wt %, to 80 wt %, to 85 wt %, to 90 wt %, to 95 wt %, to 98 wt %, to 99 wt % of the lowMW polymer in the treated material is spatially located within the upper 20%, by thickness, of the highMW polymer portion. In alternate embodiments, at least 75 wt %, to 80 wt %, to 85 wt %, to 90 wt %, to 95 wt %, to 98 wt %, to 99 wt % of the lowMW polymer in the treated material is spatially located within the upper 15%, by thickness, of the highMW polymer portion. In alternate embodiments, at least 75 wt %, to 80 wt %, to 85 wt %, to 90 wt %, to 95 wt %, to 98 wt %, to 99 wt % of the lowMW polymer in the treated material is spatially located within the upper 10%, by thickness, of the highMW polymer portion. In alternate embodiments, at least 75 wt %, to 80 wt %, to 85 wt %, to 90 wt %, to 95 wt %, to 98 wt %, to 99 wt % of the lowMW polymer in the treated material is spatially located within the upper 5%, by thickness, of the highMW polymer portion. In alternate embodiments, at least 75 wt %, to 80 wt %, to 85 wt %, to 90 wt %, to 95 wt %, to 98 wt %, to 99 wt % of the lowMW polymer in the treated material is spatially located within the upper 2%, by thickness, of the highMW polymer portion. In alternate embodiments, at least 75 wt %, to 80 wt %, to 85 wt %, to 90 wt %, to 95 wt %, to 98 wt %, to 99 wt % of the lowMW polymer in the treated material is spatially located within the upper 1%, by thickness, of the highMW polymer portion.

In at least certain embodiments wherein a film or scaffold comprising highMW polymer is surface modified (by solubilization) as described herein with a solution of lowMW amine- and/or carboxyl-terminated polymer (“lowMW polymer”), to form a treated film or scaffold (“treated material”), the lowMW polymer is dissolved in a treatment solution of 100% organic solvent (e.g., acetone, tetrahydrofuran) or a water-miscible organic solvent (“solvent”) mixture, such as acetone and water mixed in a ratio of, e.g., 99.9 vol % to 50 vol % solvent to 0.1 vol % to 50 vol % water (e.g., at least 99:1 solvent:water; at least 95:5 solvent:water; at least 90:10 solvent:water; at least 85:15 solvent:water; at least 80:20 solvent:water; at least 75:25 solvent:water; at least 70:30 solvent:water; at least 65:35 solvent:water; at least 60:40 solvent:water; at least 55:45 solvent:water; or at least 50:50 solvent:water). Optionally, the solution may comprise dimethylsulfoxide for example, in an amount of 0 vol % to 10 vol % of the treatment solution. The amount of lowMW polymer dissolved in the treatment solution may be, for example, greater than or less than 0.2 g/L of treatment solution. The time length of the incubation of the film or scaffold with the treatment solution may be in a range of 6 to 12 to 18 to 24 to 30 hours, for example. After the surface modification step with the lowMW polymer, the scaffold is optionally exposed to water to “quench” the reaction thereby enhancing the physical entrapment of the lowMW polymer within the solubilized higMW polymer component.

After a linker molecule, such as but not limited to, N-succinimidyl 3-(2-pyridyldithio) propionate (SPDP), is linked to the lowMW polymer on the surface of the highMW polymer scaffold, a cell surface binding (cell adhesion) molecule is reacted with the treated scaffold to connect the cell adhesion molecule to the scaffold for enhancing the scaffold's adhesiveness to cells applied to the scaffold in a subsequent seeding step. Molecules able to enhance binding of cells to a surface are well known in the art. Non-limiting examples of cell surface binding molecules that can be used include the cell adhesion peptides RGD, RGDC, RGDS, RGDV, RGDT, KRGD, and other RGD-containing peptides, LDV, REDV, DGEA, and glycosaminoglycans such as heparin, hyaluronic acid and chondroitin sulfate, and proteins and peptides such as cadherins, laminin, collagen, vitronectin, fibronectin, elastin, tenascin, aggrecan, and agrin, and peptide derivatives thereof such as, but not limited to, RU-1, RX-1, GD-1, GD-2, GD-3, GD-6, HGD-6, SGD-6, HSGD-6, AG-1, and F17, and tetracycline. Examples of cells that can be attached to the scaffold include, but are not limited to, mesenchymal stem cells, fibroblasts, keratinocytes, osteoblasts, osteoclasts, tenocytes, and chondrocytes, adipose-derived cells, and cells from cancer cell lines, such as but not limited to, breast cancer, prostate cancer, melanoma, colon cancer, bladder cancer, bone cancer, and any other cancer cell type for which drug testing is desired.

### EXAMPLES

Certain embodiments of the present disclosure will now be discussed in terms of several specific, non-limiting, examples. The examples described below will serve to illustrate the general practice of the present disclosure, it being understood that the particulars shown are merely exemplary for purposes of illustrative discussion of particular embodiments of the present disclosure only and are not intended to be limiting of the claims of the present disclosure.

### Example 1—Formation of Poly(L-Lactic Acid) (PLLA) Scaffold Films Bearing Cell Adhesion Peptides

In this non-limiting example of the present work, PLLA was used to produce scaffolds, which were prepared using two different processes: (1) bulk preparation, and (2) surface modification. In alternate embodiments, the processes can be conducted with any of the lowMW and highMW polymers described herein.

In the bulk preparation process for forming PLLA scaffolds, low molecular weight (lowMW) amine-terminated PLLA polymers (AT-PLLA) and/or low MW carboxy-terminated PLLA polymers (lowMW CT-PLLA) are mixed with higher MW PLLA (highMW PLLA) polymers. The highMW PLLA was obtained from Nature works LLC (average MW 50,000-100,000 Da). In the bulk preparation process, both the lowMW AT-PLLA (and/or lowMW CT-PLLA) and the highMW PLLA are dissolved in chloroform together, e.g., at concentrations of 0.01 g/mL and 0.09 g/mL respectively (e.g., a 1:9 mass/mass ratio of lowMW to highMW polymer). Then films were made by pouring the solution into 35 mm dishes to form a liquid film and dried by evaporation over 24 hours. The dried PLLA scaffold materials were removed from the dishes and kept in a pressurized chamber until use, as indicated above for the surface modification process.

In the surface modification process for forming PLLA scaffolds, scaffolds (films) were produced by dissolving highMW PLLA pellets in chloroform at a concentration of 0.1 g/mL and then poured into 35 mm dishes to form a liquid film and dried by evaporation over 24 hours. The dried highMW PLLA scaffold materials were removed from the dishes and kept in a pressurized chamber until use. The highMW PLLA was obtained from Nature Works LLC (average MW 50,000-100,000 Da). In the actual surface modification step, the upper surface of the dried highMW PLLA scaffolds were semi-permeabilized (e.g., by partial solubilization using an organic solvent such as acetone), then treated with lowMW AT-PLLA (e.g., Sigma Aldrich; average MW 2500) wherein the lowMW AT-PLLA became physically entrapped on the surface of the highMW PLLA films. In one embodiment, to solubilize the scaffold surface for incorporation of the low MW AT-PLLA to form the PLLA films, which were rinsed with phosphate-buffered saline (PBS) then incubated in 1 mL of 70% acetone in water for 1 h with moderate shaking. The acetone mixture was then aspirated and films were incubated in 1 mL of DMSO with 0.2 mg/mL lowMW AT-PLLA for 12 h again with moderate shaking. After that, films were again rinsed with PBS and left to vacuum dry for 24 h. (In alternate embodiments lowMW CT-PLLA can be used in place of, or in combination with, the lowMW AT-PLLA).

The bulk prepared and surface modified PLLA scaffolds comprising lowMW AT-PLLA (and/or lowMW CT-PLLA) were then functionalized through the chemical linking of a linker, which in this non-limiting example was N-succinimidyl 3-(2-pyridyldithio) propionate (SPDP, e.g., obtained from Thermo Scientific) to produce linker-treated scaffolds. The N-succinimidyl 3-(2-pyridyldithio) propionate (SPDP) bound to the lowMW AT-PLLA molecules via an amide bond at the amino-terminal end of the AT-PLLA molecules forming linker-modified PLLA scaffolds. To produce the linker-modified scaffolds, the films were rinsed with phosphate buffered saline (PBS) then incubated at room temperature on a shaker in 1 mL of 0.04 mmol/mL SPDP in DMSO diluted with PBS to 0.01 mmol/mL SPDP with a pH of 7.4 for 30 min.

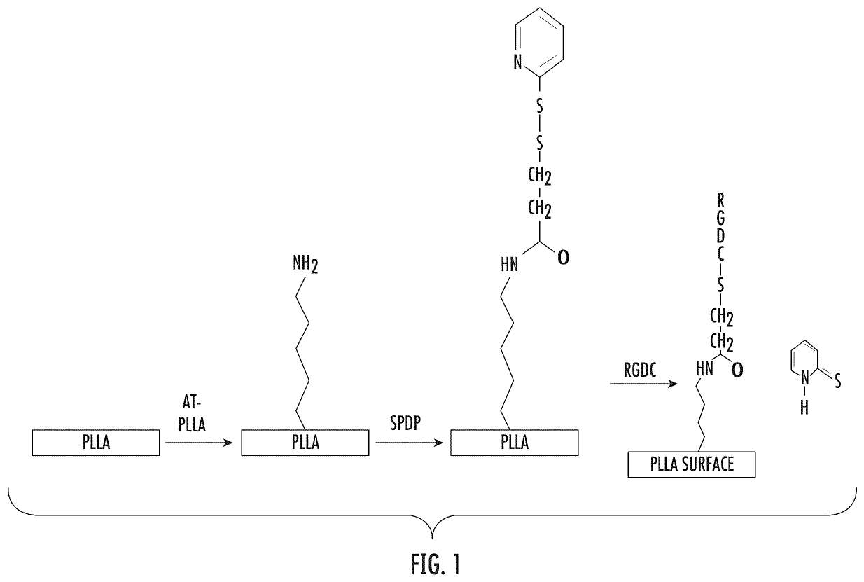

The linker-modified PLLA scaffolds (both types) were then exposed to a cell surface ligand peptide, such as but not limited to, arginine-glycine-aspartic acid-cysteine (RGDC) peptide. SPDP solution was aspirated from the linker-modified scaffolds and were incubated at 40° C. in 1 mL of 0.045 mg/mL RGDC in HEPES solution with a pH of 8.3 for 90 min. Films were then rinsed with PBS and left to vacuum dry for 24 hours before chemical sterilization. The surface modification process of adding lowMW AT-PLLA, followed by linker treatment, followed by peptide ligand treatment, is schematically represented in FIG. 1.

Amine surface-coverage and RGD-binding of the treated scaffolds were analyzed to validate the completion and longevity of the surface modification process.

### Fluorescent Scaffold Surface Amine Analysis

NHS rhodamine was used to determine the surface concentration of primary amine groups on films produced by lowMW AT-PLLA surface modification or bulk preparation. Films were incubated at room temperature on a shaker plate in 1 mL of 0.2 mg/mL NHS rhodamine in PBDS for 30 minutes. They were then rinsed with 15 min periods on the shaker plate in 1% PBST followed by two DIH₂O rinses. Each film was imaged on a Nikon Eclipse E800 microscope. Fluorescent intensity was quantified using ImageJ image analysis software. Surface modified films were compared to bulk prepared films as a control for surface presence and distribution of lowMW AT-PLLA.

### Spectrophotometric RGDC Binding Analysis

The SPDP linkage with RGDC was confirmed using spectrophotometry of the HEPES media following the RGDC incubation period. As the reaction proceeds, pyridine 2-thione is released from the SPDP molecule.

### Results and Discussion

### Scaffold Modification Validation

Validation of the physical entrapment of the lowMW AT-PLLA on the surface of the PLLA scaffolds was performed initially using the NHS rhodamine fluorescent probe for primary amine groups. Films modified with 0.2 mg/mL lowMW AT-PLLA in DMSO and unmodified PLLA films were imaged with and without NHS rhodamine treatment following 7 days of vacuum storage. The modified film displayed pervasive fluorescence while the control film displayed mild dispersed fluorescence indicative of low-level physisorption of the NHS rhodamine probe. Neither untreated scaffold demonstrated fluorescence.

Modification of 3D scaffolds was also observed using 3D-printed PLLA scaffolds. Images indicate similar surface coverage of the surface modified scaffold and minor physisorption on the unmodified scaffold (data not shown). A bright field image of the unmodified scaffold was taken as reference for the structure of the 3D-printed scaffold.

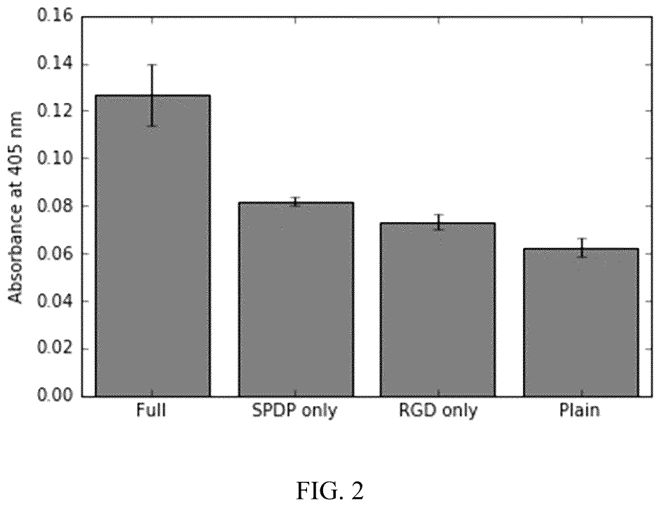

Completion of the surface modification process was confirmed using spectrophotometric analysis of the solutions used in the process (FIG. 2). For each solution, six samples were taken (n=6). The first solution was a 1 ml PBS rinse of a lowMW AT-PLLA modified film (“Plain”). The second solution was 1 ml of RGDC peptide in HEPES solution exposed to a non-linker treated lowMW AT-PLLA modified film (“RGD only”). The third solution was 1 mL of the SPDP solution exposed to a non-ligand treated lowMW AT-PLLA modified film (“SPDP only”). The fourth solution was 1 mL of RGDC in HEPES solution exposed to an SPDP-modified lowMW AT-PLLA film (“Full”). This solution displayed a significant increase in absorbance (p<0.01) as indicated by the release of pyridine 2-thione when SPDP reacts with the sulfhydryl in the RGDC peptide.

### Scaffold Modification Longevity

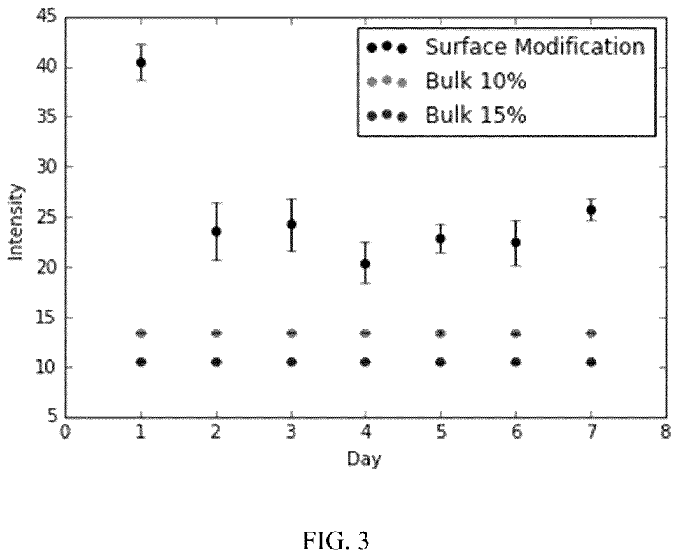

For 7 days following lowMW AT-PLLA surface modification, scaffold samples were analyzed for fluorescent intensity to determine the longevity of the physical entrapment (FIG. 3). Samples displayed fluorescent intensity within 20-25 units with no significant decrease as time progressed. With vacuum preservation, the initial physical entrapment of lowMW AT-PLLA maintained consistent surface coverage for a period of a week.

In this example, a physical entrapment surface amination technique was demonstrated for PLLA scaffolds that took advantage of PLLA's partial solubilization in acetone. We used lowMW amine-terminated PLLA (lowMW AT-PLLA), a simple molecule with a single functional primary amine attached to a short chain of PLLA. By introducing lowMW AT-PLLA during the acetone soak, a step of soaking the film with DMSO was eliminated. Use of DMSO may have resulted in physical entrapment of DMSO molecules and supported further undesirable surface modifications. DMSO has also been effectively removed from the subsequent covalent modifications by the use of an acetone+SPDP crosslinker soak. The physical entrapment method of surface modification of PLLA scaffolds results in thorough coverage on both 2D films and 3D scaffolds. In vacuum conditions, this modification resists degradation up to 7 days. Significant binding of the desired protein takes place on this surface.

### Example 2—Formation of 3-Dimensional (3D) PLLA Scaffolds with Cell Adhesion Peptides

Tissue engineering aims to utilize biomaterials, growth factors, and cells (often adult stem cells) to regenerate damaged tissue or tissue that has been removed due to cancer. The ability to seed and culture in vitro adult stem cells often of mesenchymal origin (MSC) presents unique challenges due to the inert nature of commonly used polymeric or ceramic biomaterials. Unmodified PLLA scaffolds provide consistent cellular proliferation for both 2D and 3D MSC cultures, but low initial cell attachment rates result in excessive biomass loss. In the present example, 2D PLLA films and 3D scaffolds were modified with amine-terminated PLLA molecules which were then crosslinked to arginine-glycine-aspartic acid (RGD) peptides using N-succinimidyl 3-(2-pyridyldithio) propionate (SPDP), as in Example 1. Amine surface-coverage and RGD-binding were analyzed using fluorescent and spectrophotometric chemical markers to validate the extent and longevity of the surface modification process. In alternate embodiments, the processes of this example can be conducted with any of the lowMW and highMW polymers described herein.

### Materials & Methods

### Film Preparation

2D polymer films were prepared by dissolving high MW PLLA (Nature Works LLC; average MW of 100,000) pellets in chloroform (Sigma-Aldrich). PLLA pellets were dissolved in chloroform at a concentration of 0.1 g/mL and then poured into either 35 mm or 75 mm dishes to form a thick or thin liquid film, respectively. These liquid films were then allowed to dry over 24 hours. Once dry, films were removed from the dishes and stored under vacuum until needed.

### 3D Scaffold Manufacturing

3D nonwoven fiber mesh scaffolds were produced from Poly(L-lactic acid) (PLLA; grade 6251D; 1.4% D enantiomer; 108,500 MW; 1.87 PDI; NatureWorks LLC) via spunbonding using the method taught in VanGordon et al. 2011 (VanGordon, S. B., Voronov, R. S., Blue, T. B., Shambaugh, R. L., Papavassiliou, D. V., and Sikavitsas, V. I., Effects of Scaffold Architecture on Preosteoblastic Cultures under Continuous Fluid Shear. Industrial & Engineering Chemistry Research, 50(2):620-629. 2011). Scaffolds were cut from a 5 mm thick non-woven sheet with an 8 mm diameter circular die. Individual fibers were optically analyzed using a Nikon HFX-II microscope to determine the average fiber diameter, which was found to be 24.5 μm. Twelve ï¬ber diameters were taken and averaged for each sample. The average diameter of the nonwoven ï¬bers was 24.5 μm. Finally, using an 8-mm-diameter die, disks were punched from the ï¬ber sheets. The resultant scaffolds were around 88% porous, 8 mm in diameter, and around 16 mm thick when dry. The thickness decreased down to 4.6 mm when scaffolds were wet by media. The dry volume of each scaffold was around 804 mm³. When these scaffolds were wet and inserted into the cassettes, their volume decreased down to around 231 mm³. The pore size was determined in previous studies to be around 250 μm, and was confirmed by scanning electron microscopy.

3D printed scaffolds were designed custom to provide maximum nutrient penetration. 10×10×0.54 mm square prisms were designed (SolidWorks, Waltham, MA), and sliced to produce a 65% porous fiber network (Simplify3D). Following slicing, scaffolds were printed at the following conditions: extruder temperature (215° C.), print head speed (150 mm/s), and a layer height (0.10 mm) on a Makerbot 5Error! Generation Replicator (Makerbot, Inc). Scaffolds were printed on Kapton tape (McMaster-Carr) to allow adhesion to the build plate during printing and easy removal after post-curing. Pore size and fiber diameter were measured using the aforementioned techniques.

### Modification Process

Film production by bulk preparation began with dissolving both lowMW AT-PLLA (Sigma Aldrich; average MW 2500) and highMW PLLA into chloroform at concentrations of 0.01 g/mL and 0.09 g/mL respectively. Then films were made in either 35 mm or 75 mm dishes as outlined above. Film production by surface modification began with pure high MW PLLA films (see Example 1). Films were rinsed with phosphate-buffered saline (PBS) then incubated in 1 mL of 70% acetone in water for 1 h with moderate shaking, while scaffolds were modified in 2 mL. The acetone mixture was then aspirated and films were incubated in 1 mL of DMSO with 0.2 mg/mL lowMW AT-PLLA for 12 h again with moderate shaking, with scaffolds again being incubated in 2 mL. After that, films and scaffolds were rinsed with DI H₂O and left to vacuum dry for 24 h. The water rinse insures the low MW AT-PLLA is caught in the surface of the highMW PLLA.

The bulk prepared and surface modified PLLA scaffolds comprising lowMW AT-PLLA (and/or lowMW CT-PLLA) were then functionalized through the chemical linking of a linker, which in this non-limiting example was N-succinimidyl 3-(2-pyridyldithio) propionate (SPDP, e.g., obtained from Thermo Scientific) to produce linker-treated scaffolds. The N-succinimidyl 3-(2-pyridyldithio) propionate (SPDP) bound to the lowMW AT-PLLA molecules via an amide bond at the amino-terminal end of the AT-PLLA molecules forming linker-modified PLLA scaffolds. To produce the linker-modified scaffolds, the films were rinsed with phosphate buffered saline (PBS) then incubated at room temperature on a shaker in 1 mL of 0.04 mmol/mL SPDP in DMSO diluted with PBS to 0.01 mmol/mL SPDP with a pH of 7.4 for 30 min.

The linker-modified PLLA scaffolds (both types) were then exposed to a cell surface ligand peptide, such as but not limited to, arginine-glycine-aspartic acid-cysteine (RGDC) peptide (e.g., obtained from Bachem). SPDP solution was aspirated from the linker-modified scaffolds and were incubated at 40° C. in 1 mL of 0.045 mg/mL RGDC in HEPES solution with a pH of 8.3 for 90 min. Films were then rinsed with PBS and left to vacuum dry for 24 hours before chemical sterilization.

### Fluorescent Surface Amine Analysis

NHS rhodamine was used to determine the surface concentration of primary amine groups on the films following initial treatment with lowMW AT-PLLA Films were incubated at 25° C. on a shaker plate in 1 mL of 0.2 mg/mL NHS rhodamine in PBDS for 30 minutes. They were then rinsed for 15 min periods on the shaker plate in 1% PBST followed by two DI H₂O rinses. Each film was imaged on a Nikon Eclipse E800 microscope. Fluorescent intensity was quantified using ImageJ image analysis software. Surface modified films were compared to bulk prepared films as a control for surface presence and distribution of lowMW AT-PLLA.

### Spectrophotometric RGDC Binding Analysis

The SPDP linkage with RGDC was confirmed using spectrophotometry of the HEPES media following the RGDC incubation period. As the reaction proceeds, pyridine 2-thione is released from the SPDP molecule.

### Cell Expansion and Seeding

Adult MSCs were extracted from tibias and femurs using well-established methods from male Wistar rats (Harlan Laboratories). MSCs were isolated from marrow by culturing homogenized marrow suspension in T75 cell culture flasks (Corning) for a period of three days then rinsing the flasks with PBS (Invitrogen) to remove all dead and unattached cells. These passage 0 cells were cultured at 37° C., 95% relative humidity, and 5% CO₂ in α-minimum essential medium (α-MEM; Invitrogen) supplemented with 10% fetal bovine serum (Atlanta Biologicals) and 1% antibiotic-antimycotic (Invitrogen). Media changes occurred every two days until reaching 70% confluency at which time cells were passaged (through passage 2). Passage 2 cells were lifted and suspended in α-MEM at a density of 1 million cells/mL for scaffold seeding once the culture reached 85% confluency.

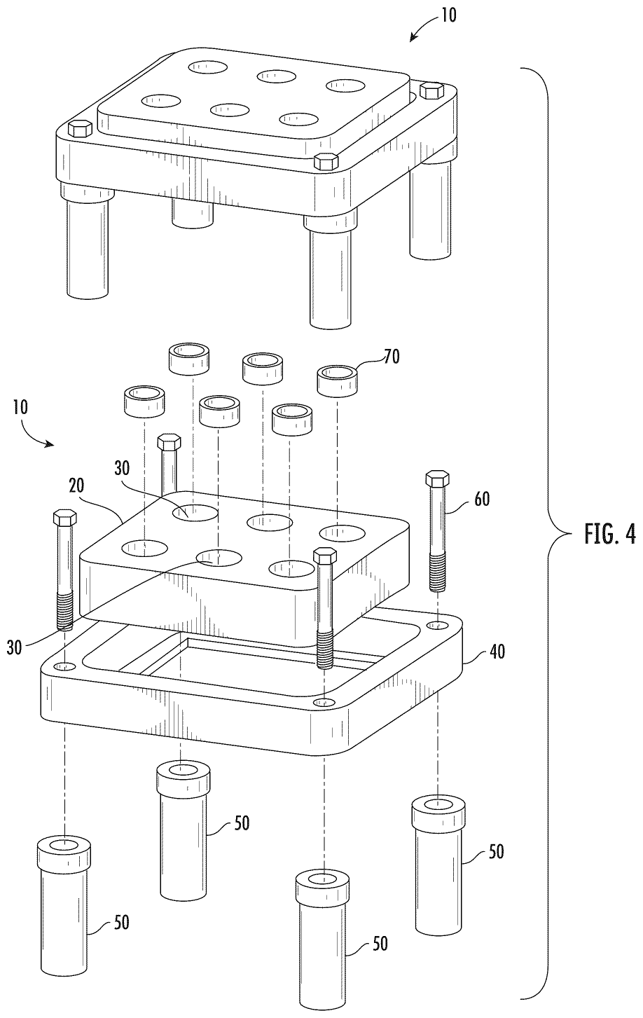

A schematic of a flow perfusion apparatus (designated by the general reference numeral 10) used for the present work is shown in FIG. 4. The flow perfusion apparatus was constructed with a scaffold cassette 20 having a plurality of scaffold receiving holes 30. The scaffold cassette 20 is placed into a cassette support 40, which is supported by a plurality of support legs 50, attached to the cassette support 40 by a plurality of fasteners 60. Scaffolds 70 are then inserted into the scaffold receiving holes 30 of the scaffold cassette 20, and the flow perfusion apparatus 10 is then placed into a vessel (not shown) where the scaffolds 70 are exposed to a perfusion medium.

To facilitate seeding, we utilized a pre-wetting technique comprising submerging scaffolds in a beaker containing 75% ethanol, placing a rubber stopper over the opening, and pulling a vacuum using a standard syringe. Pre-wetted scaffolds were then immobilized within the scaffold cassettes 20 and subsequently placed within a flow perfusion bioreactor 10 and exposed to perfusion of α-MEM for one hour prior to seeding. One million MSCs in 150 μL of α-MEM were pipetted on top of each scaffold and perfused directly through the scaffold in alternating directions for a total of two hours with a period of five minutes. Constructs were allowed to rest for an additional 2 hours before removal from the flow perfusion reactor 10.

### Construct Cellularity

The viability of cell seeded constructs sacrificed at different time points was determined using quantification of dsDNA by utilizing fluorescent PicoGreen® dsDNA assay (Invitrogen). Scaffolds were removed from cassettes, rinsed in PBS, and torn apart and submerged in 1 mL of DI water. Samples were then subjected to three freeze/thaw cycles in order to lyse the cells. 43 μl volumes for each sample were pipetted into an opaque 96-well plate (Corning) alongside standards over the assay range from 0.1 to 3 g/mL. 257 μl of buffered PicoGreen® dye was then added to each well and allowed to incubate for 5 min at 25° C. After incubation, the plate was read on a Synergy HT Multi-Mode Microplate Reader (Bio-Tek) at an excitation wavelength of 480 nm and an emission wavelength of 520 nm. All samples and standards were run in triplicate.

### Fluorescent Nucleus and Actin Staining

Three scaffolds at each time point were subjected to hoechst and phallacidin staining. This was done to confirm the cellularity result from the above dsDNA assay as well as to provide information on cell distribution within the scaffold in addition to matrix deposition. Scaffolds were resected from culture, rinsed with PBS, and fixed in solution of 4% formalin in PBS for 15 minutes at 25° C. Following this, samples were rinsed in three consecutive PBS washes. They were then permeabilized in a solution of 0.5% PBST (PBS and Triton X-100) for 15 minutes at 25° C. Fixed and permeabilized samples were stained using hoechst 33342 and phalloidin (ReadyProbes NucBlue and ActinGreen 488; Thermofisher Scientific) using manufacturer protocols. After incubation, scaffolds were rinsed thoroughly with PBS before imaging on a Nikon Epifluorescence microscope. Image analysis was performed with MetaMorph 6.2 (Universal Imaging Corporation) and Image J software packages.

### SEM Preparation and Imaging

Samples were prepared for SEM imaging by rinsing them in PBS after removing from culture. These samples were then fixed overnight in 4% formalin at 25° C. Following fixation, samples were rinsed in successive dilutions of ethanol ranging from 70% to 100%. Samples were then removed from solution and allowed to dry for 48 hours under vacuum. After mounting to SEM mounts, the samples were sputter-coated in gold palladium using a Hummer VI Triode Sputter Coater (Anatech Ltd.). SEM images were produced using a Zeiss 960 scanning electron microscope (SEM, Carl Zeiss SMT Inc) at 15 kV. Digital images were captured using EDS 2006 and EDS 2008 digital imaging software (IXRF Systems).

### Statistical Analysis

A one-way analysis of variance (ANOVA) was used to compare mean±standard deviation of pore and fiber measurements, in which Tukey's Honestly Significant Difference (HSD) test was performed to identify significant differences (p-value<0.05). One-way ANOVA and Tukey's HSD were used to compare the rest of the data. All statistical analysis was performed using a custom python code utilizing the open source Numpy, matplotlib, and SciPy libraries.

### Results

### Surface Activity

Validation of the physical entrapment of the lowMW AT-PLLA on the surface of the films was performed initially using the NHS rhodamine fluorescent probe for primary amine groups. Films modified with 0.2 mg/mL lowMW AT-PLLA in DMSO and unmodified PLLA films were imaged with and without NHS rhodamine treatment following 7 days of vacuum storage. Unmodified films showed a statistically negligible amount of fluorescence. The modified film displayed pervasive fluorescence while the control film displayed mild dispersed fluorescence indicative of low-level physisorption of the NHS rhodamine probe. Neither untreated scaffold demonstrated fluorescence. Modification of 3D scaffolds was also observed using 3D-printed PLLA scaffolds. Images indicate similar surface coverage of the modified scaffold and minor physisorption on the unmodified scaffold. A bright field image of the unmodified scaffold was taken as reference for the structure of the 3D-printed scaffold.

### Surface Modification Validation

Completion of the modification process was confirmed using spectrophotometric analysis of the solutions used in the process (FIG. 2). For each solution, six samples were taken (n=6). The first solution was a 1 ml PBS rinse of a lowMW AT-PLLA modified film (“plain”). The second solution was 1 ml of RGDC in HEPES solution exposed to a lowMW AT-PLLA modified film (“RGD only”). The third solution was 1 mL of the SPDP solution exposed to a lowMW AT-PLLA modified film (“SPDP only”). The fourth solution was 1 mL of RGDC in HEPES solution exposed to a lowMW AT-PLLA, SPDP-modified film (“Full”). The latter treatment displayed a significant increase in absorbance (p<0.01) due to the release of pyridine 2-thione when SPDP reacts with the sulfhydryl in RGDC.

### Surface Modification Longevity

For 7 days following small MW amine-terminated PLLA surface modification, scaffold samples were analyzed for fluorescent intensity to determine the longevity of the physical entrapment (FIG. 3). Samples displayed fluorescent intensity within 20-25 units with no significant decrease as time progressed. With vacuum preservation, the initial physical entrapment of small MW amine-terminated PLLA will maintain consistent surface coverage for a period of over one month.

### Surface Concentration Control

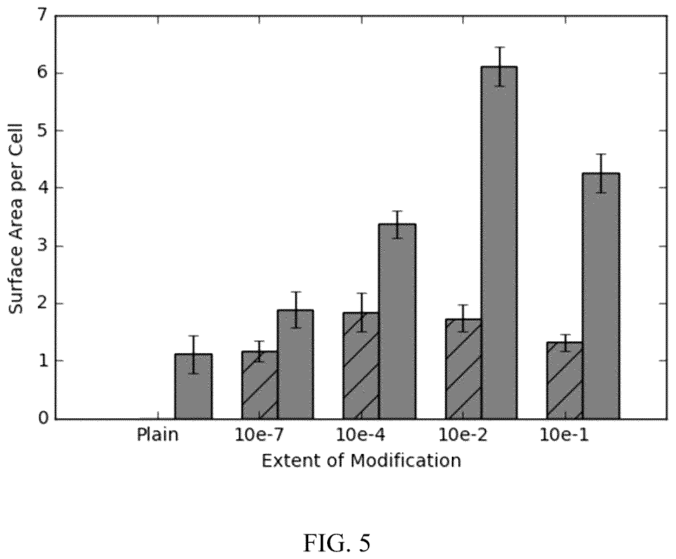

FIG. 5 compares the surface area per cell with varying surface concentrations of RGD. Error bars are shown, but are so small they are difficult to see. It is evident that there is a continual increase in cell surface area with surface area. The drop of at a concentration of 101 can be attributed to cell death due to over stimulation by RGD, which has been discussed in the literature.

### Discussion

In order to validate the functionalization process, films and 3D scaffolds were prepared that contained free terminal amine groups. The samples were incubated with NHS rhodamine, a fluorescent amine coupling tag. The control groups expressed little to physisorption indicating no free amine groups are expressed, while the amine functionalized group showed a clear fluorescence indicating free amine groups were expressed on the surface. These result show a successful amine termination scheme.

To test the viability of the second modification stage, levels of pyridine-2-thione were measured. This fluorescent moiety is released following complete SPDP conjugation. Samples were modified using the complete process: including amine termination, SPDP crosslinking, and RGD functionalization, with the samples being measured from the reaction solution. There was a statistically significant elevation of pyridine-2-thione released following RGD functionalization. This same elevation is not seen in following the intermediate steps, equating to no activation of pyridine-2-thione release. Again, this result indicates the successful binding of RGD to those aforementioned free amines.

Additionally, surface longevity of free amine expression was measured. Films and scaffolds were modified to express free amines. Those samples were then stored under vacuum for up to 7 days to allow for amine release. One week was chosen for testing due to this being the minimal time necessary for an off the shelf material to be adopted for clinical use according to our market research. Before testing, samples were rinsed in DI H₂O and dried under vacuum. Following this, samples were incubated in NHS rhodamine and imaged by fluorescent microscopy, where FIG. 3 shows the results of this process. It is evident from the results, that treatment by surface modification expresses higher amounts of amine termination over the tested time frame in comparison to films produced by bulk preparation. A large drop off between Days 1 and 2 is seen before intensity levels off. Without wishing to be bound by theory, it is believed that this decrease is due to shallow amine group penetration during the modification process, which quickly falls off. This is supported by the bulk preparation results, which do not exhibit the same initial drop off. In fact, bulk modification amine intensity levels remain constant throughout the 7 days indicating very strong binding, although both bulk levels are below those of the films produced by surface modification treatment.

Finally, the surface area per cell, or cell stretching, was investigated for varying levels of RGD modification. The surface area per cell is a measure of the actin and nucleus surface area normalized for the number of cells on the surface. This measurement is a common method of quantifying the strength of cellular binding to a surface. FIG. 5 shows an increase of cell spreading as the amount of RGD modification is increased. This fact shows that by using our modification process, we are able to tightly control the extent of RGD modification, and, by extension, the strength of cell adhesion. We attribute the decrease in cell spreading at 10⁻¹ to be associated with over stimulation of the cells resulting in cell death and detachment, which has been previously reported as a negative effect of RGD.

### CONCLUSION

In this example, we aimed to develop a physical entrapment surface amination technique for PLLA scaffolds that utilized partial solubilization in acetone of the highMW PLLA. By introducing lowMW AT-PLLA during the acetone soak, we also removed the DMSO soak which may have resulted in physical entrapment of DMSO molecules and supported further undesirable surface modifications. DMSO has also been effectively removed from the subsequent covalent modifications by the use of an acetone+SPDP crosslinker soak. The physical entrapment method of surface modification of highMW PLLA scaffolds results in thorough coverage on both 2D films and 3D scaffolds. In vacuum conditions, this modification resists degradation up to 7 days. Significant binding of the desired protein takes place on this surface. Lastly, we are able to tightly control the strength of cell adhesion to the modified surface by varying the extent of RGD functionalization.

### Example 3—N-Cadherin Mediated Enhancement of Cancer Cell Adhesion on PLLA Scaffolds Under Flow Perfusion: Development of In Vitro Tumor Models

In this example, various cancer cell lines (PC3, MDA, and B16) were seeded on 3D PLLA scaffolds, and cultured them for up to three weeks in perfusion bioreactors with samples taken intermittently. By using oscillatory seeding and surface modifications, we are able to increase seeding efficiency and, subsequently, the distribution of cells throughout the construct. Through the use of biochemical assays, fluorescent imaging, and CT we identified that these constructs behave more closely to native cancer tissue. In alternate embodiments, the processes used in this example can be conducted with any of the lowMW and highMW polymers described herein.

A promising technique for testing new drugs is to culture tumor cells in 3D polymer scaffolds in bioreactors. This method is directly analogous to tissue engineering studies, where stem cells are seeded on the scaffolds and cultured over a period of time allowing the cells to migrate, and proliferate throughout the construct. Perfusion bioreactors have proven to be the ideal method for growing these tumors due to the continuous introduction of nutrients into the system and subsequent removal of waste products. The limiting factor for this method is initial cellular adhesion. When compared to mesenchymal stem cells, cancer cells exhibit significantly lower adhesion rates. This fact means that it will take significantly longer to culture a dense tissue.

In the present example, we identified various moieties specific to certain tumors that are integral to cellular adhesion, and have used these to modify our scaffolds and trick cancer cells into exhibiting higher rates of adhesion. For instance, in terms of prostate cancer, PLLA scaffolds were modified to express n-cadherin, which is a highly upregulated protein used for cellular adhesion. N-cadherin has been shown to be very important in tumors with metastatic potential by contributing to both cell-to-cell adhesion and osteoblastic differentiation in stem cells. Various cancer cell lines (PC3, MDA, and B16) can be seeded on both 2D and 3D PLLA scaffolds, and cultured under increasing shear levels in perfusion bioreactors with samples taken intermittently. Through the use of various biochemical assays, fluorescent microscopy, and CT we identified that these constructs behave more closely to native cancer tissue in comparison to those grown using non-functionalized scaffolds. After cell seeding, we significantly increased seeding efficiency to improve cell physiology without compromising the mechanical and degradation properties of the underlying PLLA.

### Materials & Methods

### Film Preparation

PLLA films were prepared by dissolution of highMW PLLA pellets (NatureWorks LLC; average MW 100,000) in chloroform followed by evaporative deposition. PLLA pellets were dissolved in chloroform at a concentration of 0.1 g/mL and then poured into 35 mm dishes to form a liquid film and dry over 24 hours. Once dry, films were removed from the dishes and kept in a pressurized chamber until use.

### Scaffold Manufacturing

3D nonwoven fiber mesh scaffolds were produced from highMW PLLA (NatureWorks LLC, grade 6251D; 1.4% D enantiomer; 108,500 MW; 1.87 PDI) via spunbonding. Further details on the manufacturing method are found in VanGordon et al., 2011 (op. cit.). Scaffolds were cut from a 5 mm thick non-woven sheet with an 8 mm diameter circular die. Individual fibers were optically analyzed using a Nikon HFX-II microscope to determine the average fiber diameter, which was found to be 24.5 μm. Twelve fiber diameters were taken and averaged for each sample. The resultant scaffolds disks were about 88% porous, 8 mm in diameter, and about 16 mm thick when dry. The thickness decreased down to 4.6 mm when scaffolds were wet by media. The dry volume of each scaffold was about 804 mm³. When these scaffolds were wet and inserted into the cassettes, their volume decreased down to about 231 mm³. The pore size was determined in previous studies to be about 250 μm, and was confirmed by scanning electron microscopy.

3D foam scaffolds were made using solvent-cast porogen-leaching. Briefly, highMW PLLA pellets were dissolved in chloroform at a concentration of 0.1 mg/mL. NaCl was sieved to obtain grains between 250-350 μm (Sigma-Aldrich). Following this, up to 5 g of NaCl was poured in 35 μm or 75 μm glass petri dishes. After making an even bed of salt grains, the PLLA solution was poured over the bed, and the dishes were allowed to dry for 24 hours.

Following drying, the beds were divided into 2.5 g aliquots. These aliquots were placed in custom milled 8 mm mold and compressed at 500 psi. During compression, the molds were heated to 130° C., and held at constant pressure and temperature for 30 minutes. Following this process, the compressed PLLA was punched from the mold. Using a diamond saw (Model 650, South Bay Technology, Inc.), the rods were cut into 2.3 mm thick disks. The disks were placed in deionized water under agitation for 48 hours to leach out the NaCl, with DI water replaced twice daily. Following the leaching process, scaffolds were placed under vacuum to dry for 24 hours. The resulting scaffolds were 2.3 mm thick, 8 mm diameter, and about 85% porous.

### N-Cadherin Functionalization

The highMW PLLA films and scaffolds were treated by surface modification. Films were rinsed with phosphate-buffered saline (PBS) then incubated in 1 mL of 70% acetone in water for 1 h with moderate shaking, with scaffolds being incubated in 2 mL. The acetone mixture was then aspirated and films were incubated in 1 mL of DMSO with 0.2 mg/mL lowMW AT-PLLA for 12 h again with moderate shaking, and scaffolds in 2 mL, forming aminated films and aminated scaffolds. After that, the aminated films and scaffolds were again rinsed with PBS and left to vacuum dry for 24 h.

Following surface amination with the lowMW AT-PLLA, amine-amine mediated functionalization was completed through the chemical linking of SPDP and human n-cadherin (Sino Biological) to the surfaces. Films were rinsed with PBS then incubated at 25° C. on a shaker in 1 mL of 0.04 mmol/mL SPDP in DMSO diluted with PBS to 0.01 mmol/mL SPDP with a pH of 7.4 for 30 min. Scaffolds were incubated in 2 mL. SPDP solution was aspirated and scaffolds were incubated at 25° C. in 2 mL of 2.5 mg/mL n-cadherin in PBS with a pH of 7.4 for 30 min, with films being incubated in 600 μL. Scaffolds and films were then rinsed with PBS and left to vacuum dry for 24 hours before chemical sterilization.

Carboxyl-amine mediated functionalization was completed through EDC carbodiimide crosslinker (Thermo Fisher Scientific) and human n-cadherin (Sino Biological) to the surface. Briefly, n-cadherin was incubated in an activation buffer at a concentration of 1 mg/mL at 25° C. Activation buffer comprised 0.1M MES and 0.5M NaCl in PBS at a pH of 6.0. Following this, 0.4 mg EDC (˜2 mM) and 0.6 mg of NHS were added to the solution and allowed to react for 15 minutes at 25° C. Finally scaffolds were incubated in 2 mL of this solution (films in 600 μL) for 2 hours at 25° C. to facilitate complete reaction. Scaffolds and films were then rinsed with PBS and left to vacuum dry for 24 hours before chemical sterilization.

### Cell Expansion and Seeding

Adult MSCs were extracted from tibias and femurs using well-established methods from male Wistar rats (Harlan Laboratories). MSCs were isolated from marrow by culturing homogenized marrow suspension in T75 cell culture flasks (Corning) for a period of three days then rinsing the flasks with PBS (Invitrogen) to remove all dead and unattached cells. Cells were cultured at 37° C., 95% relative humidity, and 5% CO₂ in α-MEM (Invitrogen) supplemented with 10% fetal bovine serum (Atlanta Biologicals) and 1% antibiotic-antimycotic (Invitrogen). Media was changed within flasks every other day until reaching 70% confluency at which time cells were passaged (through passage 2). Passage 2 cells were lifted and suspended in α-MEM at a density of 1 million cells/mL for scaffold seeding.

PC3 prostate cells, B16 melanoma cells, and MDA breast cancer cells (ATCC) were cultured in T75 culture flasks using manufacturer recommended culture medium. PC3 and B16 cells were cultured in RPMI and MDA cells were cultured in L-15. Cells were cultured until reaching 70% confluency, and then were lifted and suspended at a density of 1 million cells/mL for scaffold seeding.

To facilitate seeding, we utilizing the pre-wetting technique described in VanGordon et al. 2011 (op. cit.). This comprises submerging scaffolds in a beaker containing 75% ethanol, placing a rubber stopper over the opening, and pulling a vacuum using a standard syringe. Pre-wet scaffolds were then immobilized within cassettes and subsequently placed within a flow perfusion bioreactor and exposed to perfusion of the cells media of choice for one hour prior to seeding. Then 1 million MSCs, PC3s, B16s, or MDAs in 150 μL of media were pipetted on top of each scaffold and perfused directly through the scaffold in alternating directions for a total of two hours with a period of five minutes. Constructs were allowed to rest for an additional 2 hours before removal.

### Construct Cellularity

The viability of cell seeded constructs sacrificed at different time points was determined using quantification of dsDNA by utilizing fluorescent PicoGreen® dsDNA assay (Invitrogen). Scaffolds were removed from cassettes, rinsed in PBS, and torn apart and submerged in 1 mL of DI water. Samples were then subjected to three freeze/thaw cycles in order to lyse the cells. 43 μl volumes for each sample were pipetted into an opaque 96-well plate (Corning) alongside standards over the assay range from 0.1 to 3 g/mL. 257 μl of buffered PicoGreen® dye was then added to each well and allowed to incubate for 5 min at 25° C. After incubation, the plate was read on a Synergy HT Multi-Mode Microplate Reader (Bio-Tek) at an excitation wavelength of 480 nm and an emission wavelength of 520 nm. All samples and standards were run in triplicate.

### Fluorescent Nucleus and Actin Staining

Three scaffolds at each time point were subjected to hoechst and phallacidin staining. This was done to confirm the cellularity result from the above dsDNA assay as well as to provide information on cell distribution within the scaffold in addition to matrix deposition. Scaffolds were resected from culture, rinsed with PBS, and fixed in solution of 4% formalin in PBS for 15 minutes at 25° C. Following this, samples were rinsed in three consecutive PBS washes. They were then permeabilized in a solution of 0.5% PBST (PBS and Triton X-100) for 15 minutes at 25° C. Fixed and permeabilized samples were stained using hoechst 33342 and phalloidin (ReadyProbes NucBlue and ActinGreen 488; Thermofisher Scientific) using manufacturer protocols. After incubation, scaffolds were rinsed thoroughly with PBS before imaging on a Nikon Epifluorescence microscope. Image analysis was performed with MetaMorph 6.2 (Universal Imaging Corporation) and Image J software packages.

### Statistical Analysis

A one-way analysis of variance (ANOVA) was used to compare mean±standard deviation of pore and fiber measurements, in which Tukey's Honestly Significant Difference (HSD) test was performed to identify significant differences (p-value<0.05). One-way ANOVA and Tukey's HSD were used to compare the rest of the results. All statistical analysis was performed using a custom python code utilizing the open source Numpy, matplotlib, and SciPy libraries.

### Results

### Surface Activity

Validation of cell seeding following modification was evaluated on 2D films. B16 cells were seeded on unmodified and modified PLLA films. Analysis showed that cells seeded on modified PLLA surfaces expressed a higher degree of cell attachment.

### Surface Modification Validation

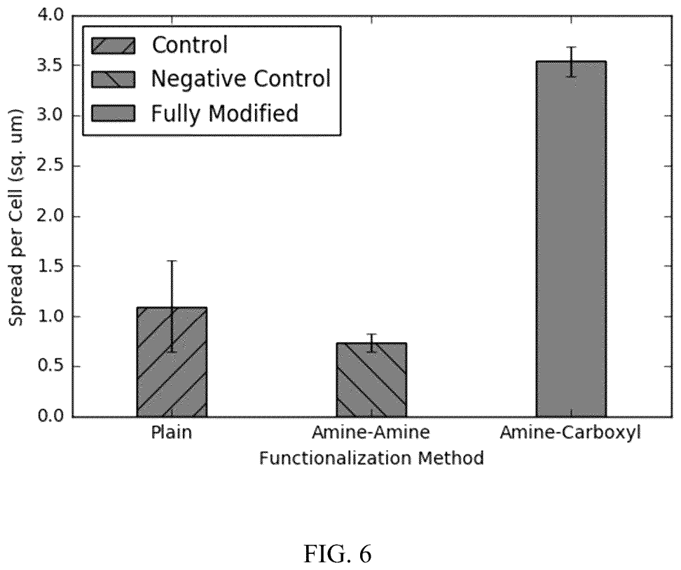

Completion of the modification process was confirmed using through the comparison of cell spreading for plain, a negative control, and a positive control (FIG. 6). Cell spreading is a measure of the surface area of actin normalized for the number of cells, and is a measure of the strength of cell adhesion. For each sample type, six samples were taken (n=6). Plain samples refer to highMW PLLA scaffolds. Negative control refers to the amine-amine reaction scheme discussed previously. This scheme produced reduced cell adhesion, despite being expected to express the n-cadherin moeity. The amine-carboxyl scheme exhibited significantly improved cell adhesion, well over two times compared to non-functionalized scaffolds.

### Seeding Efficiency

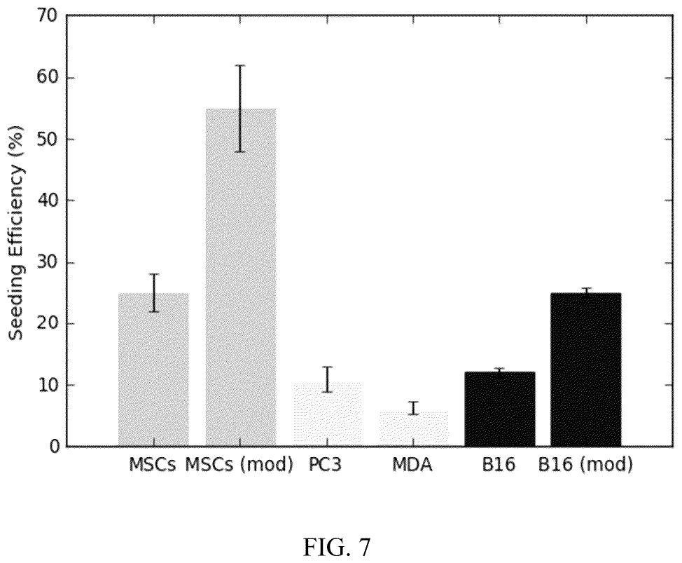

FIG. 7 shows a comparison of the seeding efficiency of cells seeded on non-modified and carboxyl modified PLLA scaffolds. It is clear that amine modified scaffolds give the highest rate of cell adhesion. On the other hand, cancer cells have highly reduced cellular seeding. With this in mind, it is a positive finding that B16 cells seeded on modified scaffolds fluorescent activity of samples with varying surface concentrations. It is evident that there is a clear increase in fluorescence as the surface concentration of increases.

### Discussion

The goal of this example was to compare the seeding efficiency of cancer cells on unmodified PLLA polymer scaffolds versus n-cadherin-modified PLLA scaffolds. Initially, a variety of cells, both MSCs and cancer, were seeded on unmodified and n-cadherin-modified 2D PLLA films. The goal of this was to gain a measure of the cell density and cell spreading on the different surfaces. After seeding, cells were fixed, stained, and imaged by fluorescent microscopy. There was a pronounced difference in B16 cell density on unmodified and modified films. Modified films showed a large amount of cells adhered to the surface of the film and elevated actin stretching, while there were minimal numbers of cells adhered to the unmodified films and minimal actin stretching.

FIG. 6 quantified difference in cellular adhesion on unmodified and modified surfaces. After generating the fluorescent images, surfaces were evaluated for the actin surface area per cell. This value gives a look into the strength of cell binding to the surface, as stronger adhesion will manifest from actin stretching out to attach to a larger surface area. As seen in the graph, cell spreading on the films modified using the amine-carboxyl modification scheme, producing stretching three times the amount of the plain and amine-amine scheme. Additionally, the amine-amine scheme functions as a negative control, as that modification process blocks binding site, preventing cells from adhering. The error present in the plain films highlights the variability exhibited when seeding cancer cells on polymer scaffolds.

Finally, the seeding efficiency for MSCs and cancer cells on 3D PLLA scaffolds which were unmodified or modified by coating with RGD peptides (as described above) was analyzed. As seen in FIG. 7, MSCs have a 30% higher adhesion rate to RGD modified scaffolds (˜55%) than unmodified (plain) scaffolds (˜25%). When comparing that result to the adhesion rates of cancer cells, it is evident that there is a large disagreement between the two. Initial seeding efficiencies on plain PLLA scaffolds for PC3s, MDAs, and B16s are about 15% lower than MSCs. MDAs exhibit the lowest adhesion rates, with less than 10% of the initial cells seeded on the scaffolds remaining after incubation. Not only is this a significant detriment in terms of the time required to culture dense tumor in vitro, but it also means that an overwhelming majority of cancer cells used for 3D in vitro culture will be wasted. Importantly, when seeded on n-cadherin coated scaffolds, the poor seeding efficiency of B16s is increased to a comparable rate of MSCs on non-modified scaffolds, over twice the initial rate. Clearly, the modification scheme presented herein provides the means to gain workable cancer cell adhesion rates on PLLA scaffolds.

As shown, cells seeded on n-cadherin expressing surfaces displayed elevated levels of adhesion. We measured the seeding efficiency of cancer cells on the two groups and compared the results with those from MSCs. By modifying the scaffolds, we were able to increase B16 seeding to rates comparable to MSCs on non-modified scaffolds. These findings support the use of the modification scheme disclosed herein to increase the viability of in vitro tumor engineered constructs.

It will be understood from the foregoing description that various modifications and changes may be made in the various embodiments of the present disclosure without departing from their true spirit. The description provided herein is intended for purposes of illustration only and is not intended to be construed in a limiting sense, except where specifically indicated. Thus, while the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus the examples described above, which include particular embodiments, serve to illustrate the practice of the present disclosure, it being understood that the particulars shown are by way of example and for purposes of illustrative discussion of particular embodiments only and are presented in the cause of providing what is believed to be a useful and readily understood description of procedures as well as of the principles and conceptual aspects of the inventive concepts. Changes may be made in the formulation of the various components and compositions described herein, the methods described herein or in the steps or the sequence of steps of the methods described herein without departing from the spirit and scope of the present disclosure.

What is claimed is:

1. A method of repairing a connective tissue defect in a subject in need of such treatment, comprising:
providing a tissue construct comprising (1) a scaffold comprising fibers of a non-water soluble high molecular weight (highMW) polymer, and (2) a partially water soluble low molecular weight (lowMW) polymer, wherein the highMW polymer has a weight average molecular weight (Mw) of at least 5,000 Da, and the partially water soluble lowMW polymer molecules (1) comprise a Mw of less than 5,000 Da, (2) comprise a terminal amine or carboxyl group, (3) are entrapped in and substantially restricted to an external surface layer of the fibers of the scaffold, and (4) comprise linker portions bound to and extending from the terminal amine or carboxyl group, the linker portions having cell adhesion molecules bound thereto, the tissue construct further comprising a plurality of cells adhered thereto via attachment to the cell adhesion molecules, wherein the cells are selected from the group consisting of osteoblasts, chondrocytes, keratinocytes, and tenocytes; and
implanting the tissue construct into the connective tissue defect, wherein the connective tissue defect is located in at least one of a bone, knee, ankle, elbow, shoulder, wrist, hip, vertebra, vertebral disc, patella, femoral head, glenoid of the scapula, growth plate, tendon, cartilage, ligament, trachea, vocal cord, bronchus, and fascia.

2. The method of claim 1, wherein the partially water soluble lowMW polymer is selected from the group consisting of polylactic acid (PLA), polyglycolic acid (PGA), poly caprolactone (PCL), poly (lactide-co-glycolide) (PLGA), poly (lactic acid/glycolic acid) (PLAGA), poly (lactide-co-caprolactone), poly (glycolide-co-caprolactone), poly (lactide-co-trimethylene carbonate), poly (glycolide-co-trimethylene carbonate), poly (lactide-co-glycolide-co-caprolactone), and poly (lactide-co-glycolide-co-trimethylene carbonate), and mixtures thereof.

3. The method of claim 1, wherein the non-water soluble highMW polymer is selected from the group consisting of polylactic acid (PLA), polyglycolic acid (PGA), poly caprolactone (PCL), poly (lactide-co-glycolide) (PLGA), poly (lactic acid/glycolic acid) (PLAGA), poly (lactide-co-caprolactone), poly (glycolide-co-caprolactone), poly (lactide-co-trimethylene carbonate), poly (glycolide-co-trimethylene carbonate), poly (lactide-co-glycolide-co-caprolactone), and poly (lactide-co-glycolide-co-trimethylene carbonate), and mixtures thereof.

4. The method of claim 1, wherein the partially water soluble lowMW polymer is a PLA selected from the group consisting of poly (L-lactic acid) (PLLA), poly (D-lactic acid) (PDLA), poly (D/L-lactic acid) (PDLLA), and poly (L/D-lactic acid) (PLDLA), and mixtures thereof.

5. The method of claim 1, wherein the non-water soluble highMW polymer is a PLA selected from the group consisting of poly (L-lactic acid) (PLLA), poly (D-lactic acid) (PDLA), poly (D/L-lactic acid) (PDLLA), and poly (L/D-lactic acid) (PLDLA), and mixtures thereof.

6. The method of claim 1, wherein at least 75 wt % of the partially water soluble lowMW polymer is spatially located within the upper 25%, by thickness, of the non-water soluble highMW polymer.
